(19) United States
(12) Patent Application Publication
(10) Pub. No.: US 2020/0360543 A1
(43) Pub. Date: Nov. 19, 2020

(54) RADIOTRACERS FOR IMAGING ER-POSITIVE BREAST CANCER

(71) Applicant: THE BOARD OF TRUSTEES OF THE UNIVERSITY OF ILLINOIS, Urbana, IL (US)

(72) Inventors: Stephen DiMagno, Chicago, IL (US); Gregory R. Thatcher, Chicago, IL (US); Debra Tonetti, Chicago, IL (US); Rui Xiong, Chicago, IL (US); Bao Hu, Chicago, IL (US)

(21) Appl. No.: 16/915,724

(22) Filed: Jun. 29, 2020

Related U.S. Application Data
(63) Continuation of application No. 16/254,017, filed on Jan. 22, 2019.
(60) Provisional application No. 62/620,097, filed on Jan. 22, 2018.

(51) Int. Cl. A61K 51/04; A61B 6/03; A61B 6/00; C07D 333/64; A61P 35/00

(52) U.S. Cl. CPC A61K 51/0431; A61B 6/037; A61B 6/502; C07B 2200/05; A61P 35/00; A61B 6/508; C07D 333/64

(57) ABSTRACT

Disclosed herein are ¹⁸F-labeled compounds and compositions thereof. Also disclosed are methods of imaging estrogen receptor expressing tissues, or methods of identifying cancer lesions using these compounds.

### CROSS-REFERENCE TO RELATED APPLICATION(S)

This application is a continuation of U.S. patent application Ser. No. 16/254,017, filed on Jan. 22, 2019, which claims the benefit of and priority to U.S. Provisional Patent Application No. 62/620,097, filed on Jan. 22, 2018, the entire contents of each of which are fully incorporated herein by reference.

### STATEMENT OF GOVERNMENT INTEREST

This invention was made with government support under grant number R01 CA188017 awarded by the National Institutes of Health. The government has certain rights in this invention.

### TECHNICAL FIELD

The present invention relates to radiolabeled compounds useful as imaging probes for estrogen receptor (ER) expressing tissues, pharmaceutical compositions thereof, and methods of using these compounds in imaging and diagnosis of diseases such as cancer.

### BACKGROUND

More women die annually of estrogen receptor positive (ER+) breast cancer than all other forms of breast cancer. Two-thirds of breast cancers are estrogen receptor positive and of these, 50% develop resistance to ER-targeted endocrine therapy, yet remain ER+ and dependent on ER for survival. Metastatic ER+ breast cancer thus poses a greater threat to women's health than all other types of breast cancer. However, metastasized tumors are very difficult to characterize and detect.

Personalized medicine is now a major focus of research and development in the pharmaceutical industry, which has a particular interest in finding the right patients for its therapies. Since 2014, more than a quarter of FDA-approved drugs comprised a personalized medication, with reference to specific biological markers that may be identified by diagnostic tools to help guide treatment decisions. Currently, more than 1,100 oncology agents are in various stages of pipeline development, and most (73 percent) are being developed inclusive of biomarkers.

Personalized treatment of metastatic disease requires early, precision identification of ER+ metastases in bone, brain, and liver. Typically, biological characterization is on tissue biopsy material after overt symptoms of recurrence. Thus, sensitive imaging techniques for detecting, identifying, and localizing metastatic cancer lesions may be essential to determine cancer recurrence, spread, and response to treatment. Localization of cancer metastasis in bone, brain, lymph, and other tissues may be achieved using Positron Emission Tomography (PET) imaging using glucose labeled with radioactive fluorine; however, this method is dependent on the higher energy requirement of cancer cells, without providing any further information on the type of metastatic cancer cells to inform treatment. An estradiol derivative labeled with radioactive fluorine (¹⁸F-fluoroestradiol, or ¹⁸F-FES) is currently being investigated for PET imaging of bone metastases in women with ER+ breast cancer (e.g., clinical trials numbers NCT01916122, NCT03266562, and NCT02409316). However, FES is endogenously rapidly metabolized and circulated causing significant background uptake in the digestive tract and liver, which complicates ER status assessment for metastatic lesions in the abdominal region.

Thus, there remains a great need for radiolabeled probes with high affinity toward ER and low background on metabolic organs, which would be useful for identifying abnormal ER status and early diagnosis of estrogen-related medical disorders such as metastatic ER+ breast cancer. Such radiolabeled probes would provide superior imaging results and would immediately inform personalized treatment with ER-directed drugs and response to treatment.

### SUMMARY

In one aspect, provided herein is a radiolabeled compound of formula (I)

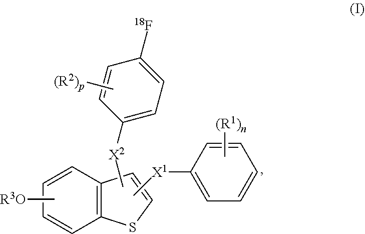

or a pharmaceutically acceptable salt thereof, wherein
X¹ is a bond, —O—, or —C(O)—;
X² is a bond, —O—, or —C(O)—;
R¹ at each occurrence is independently C₁₋₄ alkyl, C₁₋₄haloalkyl, halogen, —CN, —C(O)OR^{1a}, —(CH═CH)—C(O)OR^{1a}, —OR^{1a}, —NR^{1b}, R^{1c}, or —SO₂CH₃,
R² at each occurrence is independently C₁₋₄ alkyl, halogen, —OR^{2a}, or deuterium;
R³ is hydrogen or C₁₋₄ alkyl;
R^{1a}, R^{2a} at each occurrence are independently hydrogen, C₁₋₄ alkyl, or C₁₋₄alkylene—G¹,
R^{1b}, R^{1c} at each occurrence are independently hydrogen or C₁₋₄ alkyl;
G¹ is phenyl, a 5- or 6-membered heteroaryl, or a 5- or 6-membered heterocycle, wherein the phenyl, heteroaryl, and heterocyle are each optionally substituted with 1, 2, 3, 4, or 5 substituents independently selected from the group consisting of C₁₋₄alkyl, C₁₋₄haloalkyl, —OC₁₋₄alkyl, —C(O)—C₁₋₄alkyl, and halogen;
n is 0, 1, 2, 3, 4, or 5; and
p is 0, 1, 2, 3, or 4.

In another aspect, provided is a method of imaging a tissue expressing estrogen receptor, comprising:
administrating to a subject having an estrogen-related medical disorder a diagnostically effective amount of a radiolabeled compound disclosed herein, or a pharmaceutically acceptable salt thereof; and
detecting the radioactivity of the radiolabeled compound, or the pharmaceutically acceptable salt thereof, in the tissue of the subject.

In yet another aspect, provided is a method of identifying cancer lesion, comprising:
(a) administrating to a subject having cancer a diagnostically effective amount of a radiolabeled compound of claim 1, or a pharmaceutically acceptable salt thereof; and
(b) detecting the radioactivity of the radiolabeled compound, or the pharmaceutically acceptable salt thereof, in an organ of the subject;
wherein if the radioactivity in (b) is greater than a radioactivity in a same organ from a subject not having cancer, then the radioactivity in (b) indicates cancer lesion in the organ.

### BRIEF DESCRIPTION OF THE DRAWINGS

FIG. 1 shows a representative outline of animal imaging study. Images are generic and not meant to be reflective of the results obtained from each group.

### DETAILED DESCRIPTION

The present invention relates to compounds labeled with fluorine-18 (¹⁸F), a positron-emitting radioisotope, and the use of these compounds in diagnosis of diseases such as breast cancer. For example, these compounds may be used as positron emission tomography (PET) scan tracers for tissue imaging and for identifying cancer lesions, such as breast and lung tumors. The disclosed radiolabeled compounds may be particularly useful to identify metastatic cancer, as well as cancer amenable to endocrine and chemotherapy.

### 1. Definitions

Unless otherwise defined, all technical and scientific terms used herein have the same meaning as commonly understood by one of ordinary skill in the art. In case of conflict, the present document, including definitions, will control. Preferred methods and materials are described below, although methods and materials similar or equivalent to those described herein can be used in practice or testing of the present invention. All publications, patent applications, patents and other references mentioned herein are incorporated by reference in their entirety. The materials, methods, and examples disclosed herein are illustrative only and not intended to be limiting.

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The singular forms “a,” “and” and “the” include plural references unless the context clearly dictates otherwise. The present disclosure also contemplates other embodiments “comprising,” “consisting of” and “consisting essentially of,” the embodiments or elements presented herein, whether explicitly set forth or not.

The modifier “about” used in connection with a quantity is inclusive of the stated value and has the meaning dictated by the context (for example, it includes at least the degree of error associated with the measurement of the particular quantity). The modifier “about” should also be considered as disclosing the range defined by the absolute values of the two endpoints. For example, the expression “from about 2 to about 4” also discloses the range “from 2 to 4.” The term “about” may refer to plus or minus 10% of the indicated number. For example, “about 10%” may indicate a range of 9% to 11%, and “about 1” may mean from 0.9-1.1. Other meanings of “about” may be apparent from the context, such as rounding off, so, for example “about 1” may also mean from 0.5 to 1.4.

Definitions of specific functional groups and chemical terms are described in more detail below. For purposes of this disclosure, the chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in Organic Chemistry, Thomas Sorrell, University Science Books, Sausalito, 1999; Smith and March March's Advanced Organic Chemistry, 5^{th} Edition, John Wiley & Sons, Inc., New York, 2001; Larock, Comprehensive Organic Transformations, VCH Publishers, Inc., New York, 1989; Carruthers, Some Modern Methods of Organic Synthesis, 3^{rd} Edition, Cambridge University Press, Cambridge, 1987; the entire contents of each of which are incorporated herein by reference.

The term “alkyl” as used herein, refers to a linear or branched chain hydrocarbon containing from 1 to 10 carbon atoms. Representative examples of alkyl include, but are not limited to, methyl, ethyl, n-propyl, iso-propyl, n-butyl, sec-butyl, iso-butyl, tert-butyl, pentyl, hexyl, heptyl, octyl, nonyl and decyl.

The term “alkylene” means a divalent group derived from a saturated, straight or branched chain hydrocarbon of from 1 to 10 carbon atoms. Representative examples of alkylene include, but are not limited to, —CH₂—, —CH(CH₃)—, —C(CH₃)₂—, —CH₂CH₂—, —CH₂CH₂CH₂—, —CH₂CH₂CH₂CH₂—, —CH₂C(CH₃)₂—, and —CH₂CH(CH₃)CH₂—.

The term “fluorine” unless explicitly stated otherwise includes all fluorine isotopes. Multiple fluorine isotopes are known, however, only ¹⁹F is stable. The radioisotope ¹⁸F has a half-life of 109.8 minutes and emits positrons during radioactive decay. The relative amount of ¹⁸F present at a designated site in a compound of this disclosure will depend upon a number of factors including the isotopic purity of ¹⁸F labeled reagents used to make the compound, the efficiency of incorporation of ¹⁸F in the various synthesis steps used to prepare the compound, and the length of time since the ¹⁸F has been produced. When a position is designated specifically as ¹⁸F in the methods and compounds of the present disclosure, the position is understood to have at least 0.0001%, at least 0.0005%, at least 0.001%, at least 0.005%, at least at least 0.01%, at least 0.05%, at least 0.1%, at least 0.5%, at least 1%, at least 2%, at least 3%, at least 4%, at least 5%, at least 10%, at least 15%, at least 20%, at least 25%, at least 30%, at least 35%, at least 45%, at least 50%, at least 55%, at least 60%, at least 65%, at least 70%, at least 75%, at least 80%, at least 85%, at least 90%, at least 95%, or even at least 99% ¹⁸F incorporation at that site. These percentage values are understood to be not exceeding 99.99%. For example, the ¹⁸F incorporation at a designated position may be about 0.0001% to about 99%, about 0.001% to about 99%, about 0.01% to about 99%, about 0.1% to about 99%, about 1% to about 99%, about 25% to about 99%, about 50% to about 99%, about 75% to about 99%, or about 90 to about 99%. The ¹⁸F incorporation at a designated position may also be about 0.0001% to about 90%, about 0.0001% to about 80%, about 0.0001% to about 70%, about 0.0001% to about 60%, about 0.0001% to about 50%, about 0.0001% to about 40%, about 0.0001% to about 30%, about 0.0001% to about 20%, about 0.0001% to about 10%, about 0.0001% to about 5%, about 0.0001% to about 1%, or about 0.0001% to about 0.1%.

The term “haloalkyl” refers to an alkyl group, as defined herein, substituted by one, two, three, or four halogen atoms. Representative examples of haloalkyl include, but are not limited to, chloromethyl, 2-fluoroethyl, trifluoromethyl, pentafluoroethyl, 2-chloro-3-fluoropentyl, and 4,4,4,-trifluorobutyl.

The term “halogen” as used herein, means Cl, Br, I, or F.

The term “heteroaryl” refers to a monocyclic heteroaryl or a bicyclic heteroaryl. The monocyclic heteroaryl is a five- or six-membered ring. The five-membered ring contains two double bonds. The five-membered ring may contain one heteroatom selected from O or S; or one, two, three, or four nitrogen atoms and optionally one oxygen or sulfur atom. The six-membered ring contains three double bonds and one, two, three or four nitrogen atoms. Representative examples of monocyclic heteroaryl include, but are not limited to, furanyl, imidazolyl, isoxazolyl, isothiazolyl, oxadiazolyl, 1,3-oxazolyl, pyridinyl, pyridazinyl, pyrimidinyl, pyrazinyl, pyrazolyl, pyrrolyl, tetrazolyl, thiadiazolyl, 1,3-thiazolyl, thienyl, triazolyl, and triazinyl. The bicyclic heteroaryl includes a monocyclic heteroaryl fused to a phenyl, or a monocyclic heteroaryl fused to a monocyclic cycloalkyl, or a monocyclic heteroaryl fused to a monocyclic cycloalkenyl, or a monocyclic heteroaryl fused to a monocyclic heteroaryl, or a monocyclic heteroaryl fused to a monocyclic heterocycle. Representative examples of bicyclic heteroaryl groups include, but are not limited to, benzofuranyl, benzothienyl, benzoxazolyl, benzimidazolyl, benzoxadiazolyl, 6,7-dihydro-1,3-benzothiazolyl, imidazo[1,2-a]pyridinyl, indazolyl, indolyl, isoindolyl, isoquinolinyl, naphthyridinyl, pyridoimidazolyl, quinazolinyl, quinolinyl, thiazolo[5,4-b]pyridin-2-yl, thiazolo[5,4-d]pyrimidin-2-yl, 5,6,7,8-tetrahydroquinolin-5-yl, cyclopenta[b]thiophen-2-yl, and 4,5,6,7-tetrahydrobenzo[b]thiophen-2-yl.

The term “heterocycle” or “heterocyclyl” refers to a monocyclic heterocycle, a bicyclic heterocycle, or a tricyclic heterocycle. The monocyclic heterocycle is a three-, four-, five-, six-, seven-, or eight-membered ring containing at least one heteroatom independently selected from the group consisting of oxygen, nitrogen, phosphorus and sulfur. The three- or four-membered ring contains zero or one double bond, and one heteroatom selected from the group consisting of oxygen, nitrogen, phosphorus and sulfur. The five-membered ring contains zero or one double bond and one, two or three heteroatoms selected from the group consisting of oxygen, nitrogen, phosphorus and sulfur. The six-membered ring contains zero, one or two double bonds and one, two, or three heteroatoms selected from the group consisting of oxygen, nitrogen, phosphorus and sulfur. The seven- and eight-membered rings contains zero, one, two, or three double bonds and one, two, or three heteroatoms selected from the group consisting of oxygen, nitrogen, phosphorus and sulfur. Representative examples of monocyclic heterocycles include, but are not limited to, azetidinyl, azepanyl, aziridinyl, diazepanyl, 1,3-dioxanyl, 1,3-dioxolanyl, 1,3-dithiolanyl, 1,3-dithianyl, imidazolinyl, imidazolidinyl, isothiazolinyl, isothiazolidinyl, isoxazolinyl, isoxazolidinyl, morpholinyl, oxadiazolinyl, oxadiazolidinyl, oxazolinyl, oxazolidinyl, phosphinane, piperazinyl, piperidinyl, pyranyl, pyrazolinyl, pyrazolidinyl, pyrrolinyl, pyrrolidinyl, tetrahydrofuranyl, tetrahydropyranyl, tetrahydropyridinyl, tetrahydropyrimidinyl, tetrahydrothienyl, thiadiazolinyl, thiadiazolidinyl, thiazolinyl, thiazolidinyl, thiomorpholinyl, 1,1-dioxidothiomorpholinyl (thiomorpholine sulfone), thiopyranyl, trithianyl, and 2,5-dioxo-pyrrolidinyl. The bicyclic heterocycle is a monocyclic heterocycle fused to a phenyl group, or a monocyclic heterocycle fused to a monocyclic cycloalkyl, or a monocyclic heterocycle fused to a monocyclic cycloalkenyl, or a monocyclic heterocycle fused to a monocyclic heterocycle, or a bridged monocyclic heterocycle ring system in which two non-adjacent atoms of the ring are linked by an alkylene bridge of 1, 2, 3, or 4 carbon atoms, or an alkenylene bridge of two, three, or four carbon atoms. Representative examples of bicyclic heterocycles include, but are not limited to, benzopyranyl, benzothiopyranyl, chromanyl, 2,3-dihydrobenzofuranyl, 2,3-dihydrobenzothienyl, azabicyclo[2.2.1]heptyl (including 2-azabicyclo[2.2.1]hept-2-yl), 2,3-dihydro-1H-indolyl, isoindolinyl, octahydrocyclopenta[c]pyrrolyl, octahydropyrrolopyridinyl, 9-phosphabicyclo[3.3.1]nonane, 8-phosphabicyclo[3.2.1]octane, and tetrahydroisoquinolinyl. Tricyclic heterocycles are exemplified by a bicyclic heterocycle fused to a phenyl group, or a bicyclic heterocycle fused to a monocyclic cycloalkyl, or a bicyclic heterocycle fused to a monocyclic cycloalkenyl, or a bicyclic heterocycle fused to a monocyclic heterocycle, or a bicyclic heterocycle in which two non-adjacent atoms of the bicyclic ring are linked by an alkylene bridge of 1, 2, 3, or 4 carbon atoms, or an alkenylene bridge of two, three, or four carbon atoms. Examples of tricyclic heterocycles include, but are not limited to, octahydro-2,5-epoxypentalene, hexahydro-2H-2,5-methanocyclopenta[b]furan, hexahydro-1H-1,4-methanocyclopenta[c]furan, aza-admantane (1-azatricyclo[3.3.1.1^{3.7}]decane), oxa-adamantane (2-oxatricyclo[3.3.1.1^{3.7}]decane), and 2,4,6-trioxa-8-phosphatricyclo[3.3.1.13,7]decane.

Terms such as “alkyl,” “haloalyl,” “alkylene,” etc. may be preceded by a designation indicating the number of atoms present in the group in a particular instance (e.g., “C₁-C₄alkyl” “or “C₁₋₄alkylene”). These designations are used as generally understood by those skilled in the art. For example, the representation “C” followed by a subscripted number indicates the number of carbon atoms present in the group that follows. Thus, “C₃alkyl” is an alkyl group with three carbon atoms (i.e., n-propyl, isopropyl). Where a range is given, as in “C₁-C₄” or “C₁₋₄,” the members of the group that follows may have any number of carbon atoms falling within the recited range. A “C₁-C₄alkyl” or “C₁₋₄alkyl,”for example, is an alkyl group having from 1 to 4 carbon atoms, however arranged (i.e., straight chain or branched).

As used herein, “subject” or “patient” means an individual having symptoms of, or at risk for, a disorder or disease, such as cancer or other malignancy. A subject may be human or non-human and may include, for example, animal strains or species used as “model systems” for research purposes, such a mouse model. Likewise, subject may include either adults or juveniles (e.g., children). Moreover, subject may mean any living organism, preferably a mammal (e.g., human or non-human) that may benefit from the administration of compositions contemplated herein. Examples of mammals include, but are not limited to, any member of the Mammalian class: humans, non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like. Examples of non-mammals include, but are not limited to, birds, fish and the like. In some embodiments, the subject is a human having cancer, such as breast cancer.

The term “parenteral” or “parenterally,” as used herein, refers to modes of administration which include intravenous, intramuscular, intraperitoneal, intrasternal, subcutaneous and intraarticular injection and infusion.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

### 2. Compounds of the Invention

The present disclosure provides radiolabeled compound of formula (I)

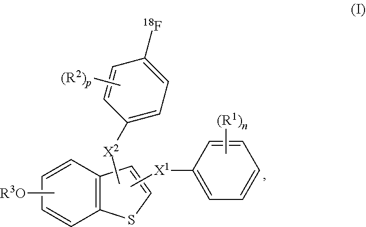

or a pharmaceutically acceptable salt thereof, wherein
X¹ is a bond, —O—, or —C(O)—;
X² is a bond, —O—, or —C(O)—;
R¹ at each occurrence is independently C₁₋₄ alkyl, C₁₋₄haloalkyl, halogen, —CN, —C(O)OR^{1a}, —(CH═CH)—C(O)OR^{1a}, —OR^{1a}, —NR^{1b},R^{1c}, or —SO₂CH₃,
R² at each occurrence is independently C₁₋₄ alkyl, halogen, —OR^{2a}, or deuterium;
R³ is hydrogen or C₁₋₄ alkyl;
R^{1a}, R^{2a} at each occurrence are independently hydrogen, C₁₋₄ alkyl, or C₁₋₄alkylene—G¹;
R^{1b}, R^{1c} at each occurrence are independently hydrogen or C₁₋₄ alkyl;
G¹ is phenyl, a 5- or 6-membered heteroaryl, or a 5- or 6-membered heterocycle, wherein the phenyl, heteroaryl, and heterocyle are each optionally substituted with 1, 2, 3, 4, or 5 substituents independently selected from the group consisting of C₁₋₄alkyl, C₁₋₄haloalkyl, —OC₁₋₄alkyl, —C(O)—C₁₋₄alkyl, and halogen;
n is 0, 1, 2, 3, 4, or 5; and
p is 0, 1, 2, 3, or 4.

In some embodiments, the compounds have formula (I-a), wherein R¹, R², R³, X¹, X², n, and p are defined as above

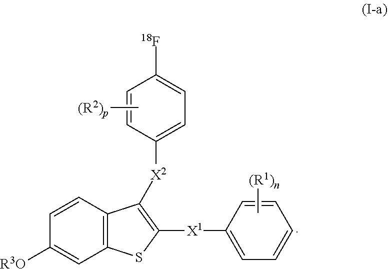

In some embodiments, X¹ is —O— or —C(O)— and X² is bond. In some embodiments, X¹ bond is and X² is —O— or —C(O)—.

In some embodiments, R¹ is C₁₋₄ alkyl, C₁₋₄haloalkyl, halogen, or —OR^{1a}. In some embodiments, R¹ is C₁₋₄ alkyl, such as methyl. In some embodiments, R¹ is halogen, such as F or Cl. In some embodiments, R¹ is —OH.

In some embodiments, R² is C₁₋₄ alkyl or halogen. In some embodiments, R² is C₁₋₄ alkyl, such as methyl. In some embodiments, R² is halogen. In some embodiments, R² is deuterium.

In some embodiments, R³ is hydrogen.

In some embodiments, n is 1, and R¹ is —OH. In some embodiments, n is 2, 3, 4, or 5, and at least one R¹ group is —OH. For example, n may be 2, 3, or 4, one R¹ group may be —OH, and the other 1, 2, or 3 R¹ groups may be independently C₁₋₄ alkyl or halogen.

In some embodiments, p is 0. In some embodiments, p is 1, 2, 3, or 4 and each R² is independently halogen or deuterium. In some embodiments, p is 1, 2, 3, or 4 and R² is deuterium.

In some embodiments, the compounds have formula (I-a-1), wherein R¹, R², R³, and p are defined as above, and n is 0, 1, 2, 3, or 4

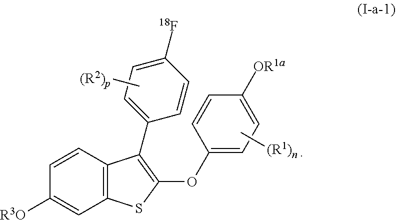

In some embodiments, the compounds have formula (I-a-1), wherein n is 0. In some embodiments, the have formula (I-a-1), wherein n is 1, 2, or, 3, and each R¹ is independently C₁₋₄alkyl.

In some embodiments, the compounds have formula (I-a-1), wherein p is 0. In some embodiments, the compounds have formula (I-a-1), wherein p is 1, 2, 3, or 4, and each R² is C₁₋₄ alkyl, halogen, or deuterium. In some embodiments, the compounds have formula (I-a-1), wherein p is 1, 2, 3, or 4, and each R² is deuterium.

In some embodiments, the compounds have formula (I-a-1), wherein R³ is hydrogen.

In some embodiments, disclosed are compounds of formula (X), or pharmaceutically acceptable salts thereof

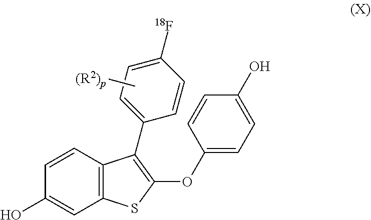

wherein R² is deuterium and p is 0, 1, 2, 3, or 4.

In some embodiments, the present disclosure provides a radiolabeled compound, which is [¹⁸F]-3-(4-(fluorophenyl)-2-(4-hydroxyphenoxy)benzo[b]thiophen-6-ol (also referred to herein as ¹⁸F-TTC-352), having a structure of

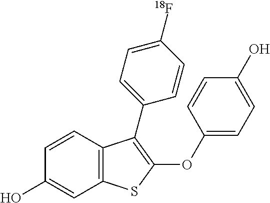 [SMILES: Oc1ccc(Oc2sc3cc(O)ccc3c2-c2ccc([18F])cc2)cc1]

or a pharmaceutically acceptable salt thereof.

The compounds disclosed herein may exist as a pharmaceutically acceptable salt. The term “pharmaceutically acceptable salt” refers to those salts which are, within the scope of sound medical judgement, suitable for use in contact with the tissues of humans and lower animals without undue toxicity, irritation, allergic response and the like, and are commensurate with a reasonable benefit/risk ratio. Pharmaceutically acceptable salts are well known in the art. For example, S. M. Berge, et al. describe pharmaceutically acceptable salts in detail in J Pharmaceutical Sciences, 1977, 66, 1-19, incorporated herein by reference in its entirety. Pharmaceutically acceptable salts of the compounds disclosed herein include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. The present disclosure also envisions the quaternization of any basic nitrogen-containing groups of the compounds disclosed herein. Water or oil-soluble or dispersable products may be obtained by such quaternization. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate and aryl (e.g., phenyl/substituted phenyl) sulfonate.

Basic addition salts may be prepared during the final isolation and purification of the present compounds, for example, by reaction of a carboxyl group with a suitable base such as the hydroxide, carbonate, or bicarbonate of a metal cation such as lithium, sodium, potassium, calcium, magnesium, or aluminum, or an organic primary, secondary, or tertiary amine. Quaternary amine salts derived from methylamine, dimethylamine, trimethylamine, triethylamine, diethylamine, ethylamine, tributylamine, pyridine, N,N-dimethylaniline, N-methylpiperidine, N-methylmorpholine, dicyclohexylamine, procaine, dibenzylamine, N,N-dibenzylphenethylamine, 1-ephenamine and N,N′-dibenzylethylenediamine, ethylenediamine, ethanolamine, diethanolamine, piperidine, piperazine, and the like, are contemplated as being within the scope of the present invention.

In some embodiments, the compounds or pharmaceutically acceptable salts thereof have a radiochemical purity of about 90% to about 99.9%. This includes radiochemical purity of about 91% to about 99.9%, about 92% to about 99.9%, about 93% to about 99.9%, about 94% to about 99.9%, about 95% to about 99.9%, about 96% to about 99.9%, about 97% to about 99.9%, about 98% to about 99.9%, or about 99% to about 99.9%. The radiochemical purity may be at least 90%, such as at least 91%, at least 92%, at least 93%, at least 94%, at least 95%, at least 96%, at least 97%, at least 98%, or even at least 99%, which value is understood to be not exceeding 99.99%. Radiochemical purity may refer to the proportion of the total radioactivity in a sample which is present as the desired radiolabeled compound.

In some embodiments, the synthesis of the present radiolabeled compounds, such as ¹⁸F-TTC-352, may use a diaryliodonium salt precursor which was previously employed for the preparation a wide variety of ¹⁸F-substituted arenes. The general methodology is outlined in Scheme 1 (top panel).

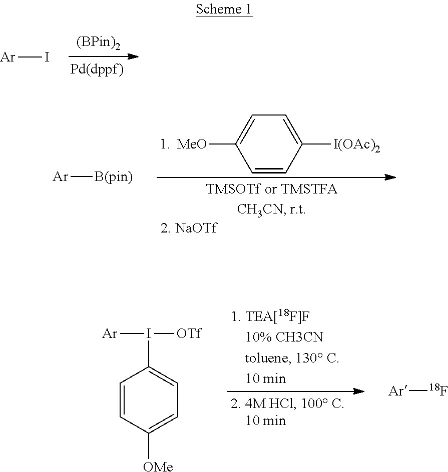

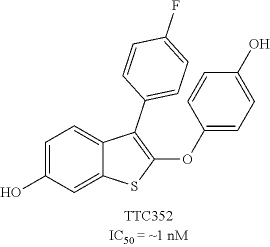 [SMILES: Oc1ccc(Oc2sc3cc(O)ccc3c2-c2ccc(F)cc2)cc1]

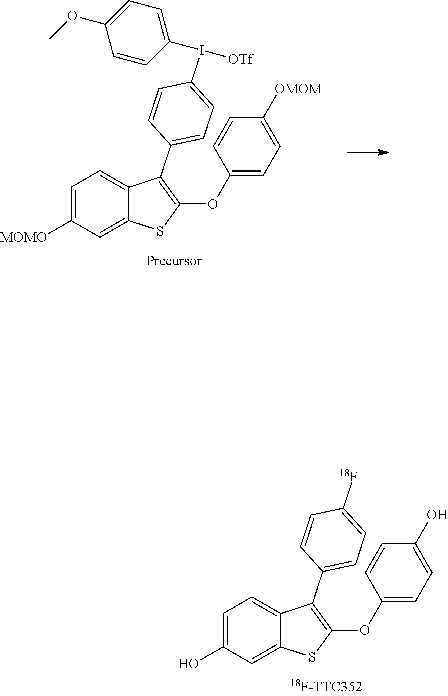

In some embodiments, a small-scale synthesis of the finished precursor and the labeling experiments may be conducted (e.g., <20% RCY, Scheme 1, bottom panel). The synthesis may be carried out under experimental conditions in automated synthesis modules that mimic those previously employed for the production of [¹⁸F]F-DA, [¹⁸F]F-DOPA and [¹⁸F]F-MFBG, for which automated scripts are available. In some embodiments, a large-scale (multigram) synthesis of the precursor may be carried out and automated synthesis may be implemented. Suitable synthesis methods include those described in U.S. Pat. No. 8,604,213, which is incorporated herein by reference in its entirety.

The synthesis of the radiolabeled compounds herein may use protecting groups known in the art. Protecting groups may be a temporary substituent which protects a potentially reactive functional group from undesired chemical transformations. The choice of the particular protecting group employed may depend on a number of considerations including, but not limited to, the functional group being protected, other functionality present in the molecule, reaction conditions at each step of the synthetic sequence, other protecting groups present in the molecule, functional group tolerance to conditions required to remove the protecting group, and reaction conditions for the thermal decomposition of the compounds provided herein. The field of protecting group chemistry has been reviewed (Greene, T. W.; Wuts, P. G. M. Protective Groups in Organic Synthesis, 2^{nd} ed.; Wiley: New York, 1991).

A nitrogen protecting group may be any temporary substituent which protects an amine moiety from undesired chemical transformations. Examples of such protecting groups include, but are not limited to allylamine, benzylamines (e.g., bezylamine, p-methoxybenzylamine, 2,4-dimethoxybenzylamine, and tritylamine), acetylamide, trichloroacetammide, trifluoroacetamide, pent-4-enamide, phthalimides, carbamates (e.g., methyl carbamate, t-butyl carbamate, benzyl carbamate, allyl carbamates, 2,2,2-trichloroethyl carbamate, and 9-fluorenylmethyl carbamate), imines, and sulfonamides (e.g., benzene sulfonamide, p-toluenesulfonamide, and p-nitrobenzenesulfonamide).

An oxygen protecting group may be any temporary substituent which protects a hydroxyl moiety from undesired chemical transformations. Examples of such protecting groups include, but are not limited to esters (e.g., acetyl, t-butyl carbonyl, and benzoyl), benzyl (e.g., benzyl, p-methoxybenzyl, and 2,4-dimethoxybenzyl, and trityl), carbonates (e.g., methyl carbonate, allyl carbonate, 2,2,2-trichloroethyl carbonate and benzyl carbonate) ketals, and acetals, and ethers.

### 3. Pharmaceutical Compositions

Compounds of the invention may be incorporated into pharmaceutical compositions suitable for administration to a subject (such as a patient, which may be a human or non-human).

The pharmaceutical compositions may include a “diagnostically effective amount” or “therapeutically effective amount” of the radiolabeled compounds disclosed herein. A “diagnostically effective amount” refers to an amount of the radiolabeled compounds, or pharmaceutically acceptable salts thereof or compositions thereof, being administered where a detectable signal is produced. For diagnosis of cancer, the “diagnostically effective amount” may include, for example, the amount of a compound or a composition required to identify the size of tumor, the extent of cancer lesion, the location of cancer metastasis, and differentiation of nascent cancer cell from normal cells. A “diagnostically effective amount” of the compounds or compositions may be determined by a person skilled in the art and may vary according to factors such as the disease state, age, sex, and weight of the individual, and the ability of the composition to elicit a desired response in the individual. A “diagnostically effective amount” may also be one in which any toxic or detrimental effects of the compounds or compositions are outweighed by the diagnostically beneficial effects.

For example, a diagnostically effective amount of a high specific activity ¹⁸F-labeled compound is generally between 1 and 10 mCi. Because this compound may be prepared in 0.1-10 Ci/micromole specific activity, the mass of a diagnostically effective amount of compound of formula (I) may be about 0.1 micrograms to about 100 micrograms for an adult human dose. In some embodiments, a diagnostically effective dose includes about 0.1 to about 90 micrograms, about 0.1 to about 80 micrograms, about 0.1 to about 60 micrograms, about 0.1 to about 40 micrograms, about 0.1 to about 20 micrograms, about 0.1 to about 10 micrograms, about 0.1 to about 8 micrograms, about 0.1 to about 6 micrograms, about 0.1 to about 5 micrograms, about 0.1 to about 4 micrograms, about 0.1 to about 3 micrograms, about 0.1 to about 2 micrograms, or about 0.1 to about 1 micrograms of a ¹⁸F-labeled compound. In particular embodiments, a diagnostically effective dose includes about 0.5 micrograms, about 1.0 micrograms, about 1.5 microgram, about 2.0 micrograms, about 2.5 micrograms, about 3.0 micrograms, about 3.5 micrograms, about 4.0 micrograms, about 4.5 micrograms, or about 5.0 micrograms of a ¹⁸F-labeled compound.

The pharmaceutical compositions may include pharmaceutically acceptable carriers. The term “pharmaceutically acceptable carrier,” as used herein, means a non-toxic, inert solid, semi-solid or liquid filler, diluent, encapsulating material or formulation auxiliary of any type. Some examples of materials which can serve as pharmaceutically acceptable carriers are sugars such as, but not limited to, lactose, glucose and sucrose; starches such as, but not limited to, corn starch and potato starch; cellulose and its derivatives such as, but not limited to, sodium carboxymethyl cellulose, ethyl cellulose and cellulose acetate; powdered tragacanth; malt; gelatin; talc; excipients such as, but not limited to, cocoa butter and suppository waxes; oils such as, but not limited to, peanut oil, cottonseed oil, safflower oil, sesame oil, olive oil, corn oil and soybean oil; glycols; such as propylene glycol; esters such as, but not limited to, ethyl oleate and ethyl laurate; agar; buffering agents such as, but not limited to, magnesium hydroxide and aluminum hydroxide; alginic acid; pyrogen-free water; isotonic saline; Ringer's solution; ethyl alcohol, and phosphate buffer solutions, as well as other non-toxic compatible lubricants such as, but not limited to, sodium lauryl sulfate and magnesium stearate, as well as coloring agents, releasing agents, coating agents, sweetening, flavoring and perfuming agents, preservatives and antioxidants can also be present in the composition, according to the judgment of the formulator.

In some embodiments, the composition is a liquid composition suitable for intravenous injection.

In some embodiments, the radiolabeled compounds or compositions may have a specific activity of at least 0.01 Ci/μmol, such as at least 0.05, at least 0.10, at least 0.20, at least 0.30, at least 0.40, at least 0.50, at least 0.60, at least 0.70, at least 0.80, at least 0.90, at least 1.00, at least 1.50, at least 2.00, at least 2.50, at least 3.00, at least 3.50, at least 4.00, or at least 4.50 Ci/μmol. In some embodiments, the radiolabeled compounds or compositions may have a specific activity of less than 5.00 Ci/μmol, such as less than 4.50, less than 4.00, less than 3.50, less than 3.00, less than 2.50, less than 2.00, less than 1.50, less than 1.00, less than 0.90, less than 0.80, less than 0.70, less than 0.60, less than 0.50, less than 0.40, less than 0.30, less than 0.20, less than 0.10, or less than 0.05 Ci/μmol. The radiolabeled compounds or compositions may have a specific activity of about 0.10 to about 5.00 Ci/μmol, such as about 0.10 to about 4.50, about 0.10 to about 4.00, about 0.10 to about 3.50, about 0.10 to about 3.00, about 0.10 to about 2.50, about 0.10 to about 2.00, about 0.10 to about 1.50, about 0.10 to about 1.00, about 0.10 to about 0.80, about 0.10 to about 0.60, about 0.10 to about 0.40, about 0.20 to about 1.00, about 0.20 to about 0.80, about 0.20 to about 0.60, about 0.50 to about 1.00, or about 0.50 to about 0.80 Ci/μmol.

### 4. Methods of Use

The present compounds may be used as radiolabeled probes for diagnosis of disorders and diseases. In particular, the present disclosure provides a companion biomarker for a selective human estrogen receptor partial agonist TTC-352, as described in described in WO2014066692A1 and WO2014066695A1, the contents of which are incorporated herein by reference in their entirety. The disclosed radiolabeled compounds (such as ¹⁸F-TTC-352) and compositions therefore may be used in methods to image estrogen-related medical disorders. The method may include administrating to a subject having an estrogen-related medical disorder a diagnostically effective amount of a disclosed radiolabeled compound, or a pharmaceutically acceptable salt thereof, or a composition thereof. The method may further include detecting the radioactivity of the radiolabeled compound, or the pharmaceutically acceptable salt thereof, or the composition thereof in a tissue of the subject. The tissue can be any tissue that expresses the estrogen receptor, such as a cancerous lesion.

### A. Imaging Estrogen-Related Disorders

In some embodiments, a method is provided for imaging a tissue expressing estrogen receptor, comprising:
administrating to a subject having an estrogen-related medical disorder a diagnostically effective amount of a radiolabeled compound of claim 1, or a pharmaceutically acceptable salt thereof and
detecting the radioactivity of the radiolabeled compound, or the pharmaceutically acceptable salt thereof, in the tissue of the subject.

An estrogen-related medical disorder may be any medical disorder in which the activity of an estrogen receptor is altered or changed. Alteration of the activity of an estrogen receptor may include upregulation or downregulation of estrogen receptor activity. Alteration of the activity of an estrogen receptor may be the same or different in organs, tissues, and/or cells of a subject.

An estrogen-related medical disorder may also be any medical disorder responsive to modulation of the activity of an estrogen receptor. Such modulation of the activity of an estrogen receptor may include upregulation or downregulation of estrogen receptor activity. The activity of an estrogen receptor may be modulated or altered by an agonist, an antagonist, a selective estrogen receptor modulator (SERM), a selective estrogen mimic (SEM), and/or derivatives thereof. The activity of the estrogen receptor may be modulated the same or differently in different organs, tissues, and/or cells of a subject.

An estrogen-related medical disorder may further be any medical disorder caused by the action of estrogen and/or lack of estrogen action. An estrogen-related medical disorder may be any medical disorder responsive or sensitive to a composition of the present invention.

An estrogen-related medical disorder may be hormone dependent or hormone independent. An estrogen-related medical disorder may include, but is not limited to, cancer, inflammation, osteoporosis, vaginal atrophy, central nervous system diseases, and cardiovascular diseases.

In some embodiments, the present imaging methods comprise administrating to a subject ¹⁸F-TTC-352,

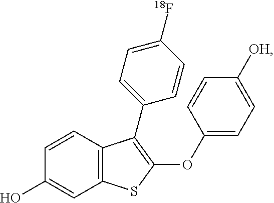 [SMILES: Oc1ccc(Oc2sc3cc(O)ccc3c2-c2ccc([18F])cc2)cc1]

or a pharmaceutically acceptable salt thereof

Typically, the present compounds and compositions may produce a detectable signal, (e.g., gamma emission) after being administered to the subject. The detectable signal may be localized in one or more organs or tissues of the subject, such as brain, breast, liver, or lung. A detectable signal, e.g., one that can be detected over the background signal, may be a signal derived from non-invasive imaging techniques such as, but not limited to, positron emission tomography (PET). The detectable signal is detectable and distinguishable from other background signals that may be generated from the subject. In other words, there is a measurable and statistically significant difference between detectable signal and the background. For example, a statistically significant difference may be enough of a difference to distinguish among the acoustic detectable signal and the background, such as about 0.1%, 1%, 3%, 5%, 10%, 15%, 20%, 25%, 30%, or 40% or greater difference between the detectable signal and the background). Standards and/or calibration curves can be used to determine the relative intensity of the detectable signal and/or the background.

In some embodiments, the present imaging method comprises detecting the radioactivity of the radiolabeled compounds, or the pharmaceutically acceptable salts thereof, by positron emission tomography (PET). Due to the presence of ¹⁸F, the compounds may be used for PET imaging. The PET imaging can be conducted for about 1 minute to about 8 hours, such as about 30 minutes to about 5 hours, 1 hour to about 4 hours, or about 1 hour to about 3 hours. In some embodiments, the PET scan is conducted for about 2 hours. In some embodiments, the duration of the PET imaging is less than 1 hour, such as about 30 minutes, about 10 minutes, about 5 minutes, about 2 minutes, about 1 minute, or about 0.5 minute.

PET images may be taken at varying time post administration of the radiolabeled compound. For example, PET images may be taken about 10 seconds to about 6 hours post administration, such as about 5 minutes to about 3 hours, about 10 minutes to about 1 hour, about 10 minutes to about 12 hours, about 5 minutes to about 6 hours, about 5 minutes to about 1 hour, or about 1 minute to about 15 minutes post-administration. The PET images may be captured post a single administration or of a course of continuous administration. In addition, PET images may be takes once a day, twice a day, or any suitable amount of times daily (within the time range post-administration listed above) that is suitable to achieve adequate detection of the radiolabeled compound.

The administration amounts discussed above for the pharmaceutical compositions can also be applied to the amount administrated for PET imaging. In some embodiments, PET images are taken at about 2 hours post administration for subjects receiving 1 mCi, 5 mCi, or 10 mCi. In some embodiments, PET images are taken at about 30 minutes post administration for subjects receiving 1 mCi, 5 mCi, or 10 mCi.

In some embodiments, a dose of about 0.1 to about 20 mCi of radiolabeled compounds or compositions disclosed herein may be administered intravenously. This includes amount of at least 0.5, at least 1, at least 2, at least 3, at least 4, at least 5, at least 6, at least 7, at least 8, at least 9, at least 10, at least 12, at least 14, at least 16, or at least 18 mCi. The amount of radiolabeled compounds or compositions may be less than 19, less than 17, less than 15, less than 13, less than 11, less than 10, less than 9, less than 8, less than 7, less than 6, less than 5, less than 4, less than 3, or less than 2 mCi. In some embodiments, the amount is about 2 to about 18 mCi, about 2 to about 16 mCi, about 2 to about 14 mCi, about 2 to about 12 mCi, about 2 to about 10 mCi, about 3 to about 10 mCi, about 4 to about 10 mCi, or about 5 to about 10 mCi. The radiolabeled compounds or compositions may be administered in a liquid dose of about 1 to about 30 mL, such as about 10 to about 30 mL or about 10 to about 20 mL. The liquid dose may comprise an aqueous solution having a pH of about 7.4. In some embodiments, the liquid dose is an isotonic PBS solution containing less than 15% of ethanol by volume.

In addition, the disclosed compounds may allow for advantageous detection of tissues that express the estrogen receptor. For example, the disclosed compounds may be able to detect a tissue expressing the estrogen receptor at signal to noise ratio of about 2:1 to about 1000:1, such as about 2:1 to about 500:1, about 2:1 to about 100:1, about 2:1 to about 50:1, about 2:1 to about 10:1, about 2:1 to about 8:1, about 2:1 to about 5:1, or about 2:1 to about 3:1. In some embodiments, the signal to noise ratio is about 2:1 to about 10:1. In particular embodiments, the signal to noise ratio is about 3:1, about 4:1, about 5:1, about 6:1, or about 8:1.

### Cancer

The present methods may be used for imaging estrogen-related medical disorders, for example, cancer. The cancer may be a breast cancer, a uterine cancer, an ovarian cancer, a prostate cancer, and a lung cancer. In some embodiments, the present method is be used for imaging breast cancer. In some embodiments, the breast cancer is ER-positive breast cancer. In some embodiments, the breast cancer is resistant to ER-targeted endocrine therapy. Particularly, the breast cancer may be a tamoxifen resistant breast cancer.

The imaging methods may be able to determine the stage of breast cancer. For example, in some embodiments, the imaging method can stratify subjects and align them with a specific stage of breast cancer. The stages of breast cancer are based on a number of factors, such as the size of the tumor, if cancer is found in the lymph nodes, and how far the cancer has spread. The stages are numbered 0, I, II, III or IV, with Stage I being the least advanced stage and Stage IV being the most advanced. Stage 0 is considered non-invasive breast cancer. Stages I-II is considered early breast cancer. Stage III is considered locally advanced breast cancer. Stage IV is considered metastatic breast cancer. These descriptions are broad descriptions of breast cancer stages, and may not include all possibilities. In addition, the methods of imaging may be able to stratify subjects by removing potential non-responders (e.g., to a treatment regimen) from responders. For example, the imaging methods may be used to stratify subjects that have metastatic breast cancer in order to indicate whether they may respond to endocrine therapy, cytotoxic chemotherapy, molecularly targeted therapy, or a combination thereof.

### Inflammation

The present methods may be used for imaging estrogen-related medical disorders, for example, inflammation. The methods of treatment may be able to detect inflammation in a subject in need of such treatment.

### Osteoporosis

The present methods may be used for imaging estrogen-related medical disorders, for example, osteoporosis. The methods of treatment may be able to detect osteoporosis in a subject. The methods of treatment may detect the loss of bone mineral density in a subject. The present imaging methods may detect the rate of bone turnover or fractures. The present imaging methods may provide guidance for treatment plans for improving or maintaining bone mineral density or reversing osteoporosis in a subject.

### Vaginal Atrophy

The present methods may be used for imaging of estrogen-related medical disorders, for example, vaginal atrophy. The present imaging methods may detect vaginal atrophy in a subj ect.

### Cardiovascular System Diseases

The present methods may be used for imaging estrogen-related medical disorders, for example, cardiovascular system diseases. For example, the imagining methods may be able to detect certain estrogen related cardiovascular diseases, or be able to detect the stage of the cardiovascular disease. The methods of imaging may detect the vasodilatory effects of estradiol on the coronary vasculature. The present imaging methods may facilitate assessment of the risk of thromobosis, stroke, and/or coronary heart disease.

### Central Nervous System Diseases

The present methods invention may be used for imaging estrogen-related medical disorders, for example, central nervous system diseases. The disease of the central nervous system may be Alzheimer's Disease or mild cognitive impairment. The present imaging methods may detect the tissue abnormalities associated with Alzheimer's Disease or mild cognitive impairment in a subject. In addition, the methods of imaging may be able to detect varying stages of Alzheimer's Disease or cognitive impairment.

### B. Identification of Cancer Lesion

The disclosed radiolabeled compounds and compositions thereof may be used in methods of identifying cancer lesions. After administration, the radioactivity from compounds and composition may be detected in an organ of a subject having cancer such as ER positive breast cancer, which may be compared to the results from a control subject not having cancer (e.g., negative control), and an increased level radioactivity detected in the organ indicates cancer lesion the organ of the subject.

In some embodiments, the present disclosure provides a method of identifying cancer lesion, comprising:
(a) administrating to a subject having cancer a diagnostically effective amount of a radiolabeled compound of claim 1, or a pharmaceutically acceptable salt thereof; and
(b) detecting the radioactivity of the radiolabeled compound, or the pharmaceutically acceptable salt thereof, in an organ of the subject;
wherein if the radioactivity in (b) is greater than a radioactivity in a same organ from a subject not having cancer, then the radioactivity in (b) indicates cancer lesion in the organ.

In some embodiments, the cancer lesion is a result of cancer metastasis. The cancer metastasis can be localized in varying types of tissues, including, but not limited to, bone, brain, chest wall, liver, lymph, and lung. As such, in some embodiments, the cancer lesion is localized in varying types of tissues, including, but not limited to, bone, brain, chest wall, liver, lymph, and lung.

In some embodiments, the cancer is breast cancer. In particular embodiments, the breast cancer is an ER-positive breast cancer. In some embodiments, the cancer may be a recurrent cancer.

As described above, the presence of ¹⁸F isotope may allow the compounds and the pharmaceutically acceptable salts thereof to be detected by PET imaging. Accordingly, the radioactivity produced by the compounds or compositions after administration to the subject may be detected by PET. In some embodiments, the methods of identifying cancer lesion comprise detecting the radioactivity by PET under conditions (such as scan durations and schedules) similar to those used for the methods of imaging tissues expressing estrogen receptor described above for the diagnosis of estrogen-related medical disorders.

The administration amounts discussed above for the pharmaceutical compositions can also be applied to the amount administrated for PET imaging. In some embodiments, PET images are taken at about 2 hours post administration for subjects receiving 1 mCi, 5 mCi, or 10 mCi. In some embodiments, PET images are taken at about 30 minutes post administration for subjects receiving 1 mCi, 5 mCi, or 10 mCi.

In addition, the disclosed compounds may allow for advantageous detection of cancerous lesions that express the estrogen receptor. For example, the disclosed compounds may be able to detect a cancerous lesion at signal to noise ratio of about 2:1 to about 1000:1, such as about 2:1 to about 500:1, about 2:1 to about 100:1, about 2:1 to about 50:1, about 2:1 to about 10:1, about 2:1 to about 8:1, about 2:1 to about 5:1, or about 2:1 to about 3:1. In some embodiments, the signal to noise ratio is about 2:1 to about 10:1. In particular embodiments, the signal to noise ratio is about 3:1, about 4:1, about 5:1, about 6:1, or about 8:1.

In some embodiments, the method of identifying cancer lesion comprise administrating to a subject having cancer a diagnostically effective amount of the ¹⁸F-TTC-352

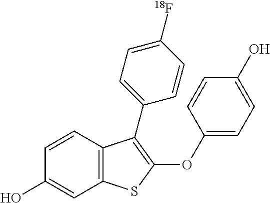 [SMILES: Oc1ccc(Oc2sc3cc(O)ccc3c2-c2ccc([18F])cc2)cc1]

or a pharmaceutically acceptable salt thereof, or a composition thereof.

In some embodiments, a PET scan using the compounds or compositions disclosed herein may be carried out to confirm the estrogen receptor status of recurrent tumors. A representative imaging schedule may be pre-therapy, 3 to 6 months post therapy, and then annually for 2-3 years. The imaging may provide clinical information, such as 1) the presence/absence of ER+tumors and micrometastases and 2) the ER status of potential lesions seen in anatomical imaging. In some embodiments, the tracer compound may not localize in tumors that are ER negative. The safety risks may be typical of any PET imaging study. In some embodiments, the tracer compounds herein are administered in extremely low dose (e.g., 1 to 100 micrograms, such as 5 to 50 micrograms or 10 to 40 micrograms), and there are not toxicological risks. Risks from radiation and dosimetry may need to be verified in the Phase I clinical trial. Typically, absorbed dose with PET is equivalent to a full body CT scan. The imaging may inform disease management, particularly in gauging the effectiveness of therapy. The imaging may also serve as a much more sensitive measure of cancer recurrence.

The methods disclosed herein may be combined with a computerized tomography (CT) scan, a magnetic resonance imaging (MM) scan, and/or PET scan with other tracers in the subject. The CT scan and MM scan may be done before or after the detection of radioactivity (e.g., PET imaging). In addition, the methods disclosed herein may be combined with ¹⁸F-FDG PET. ¹⁸F-FDG PET may allow for the detection of metabolically active tumors. Current practice for breast cancer diagnosis involves CT imaging (mammography) followed by multiple invasive, expensive, painful, and sometimes inaccurate needle biopsies following by pathological analysis to identify suspect masses. Thus, the ER-binding compounds and PET imaging methods disclosed herein may use as an alternative to biopsy for early stage (or suspected) breast cancer diagnosis. For example, PET imaging with ¹⁸F-TTC-352 in conjunction with CT and/or ¹⁸F-FDG PET, may allow confirmation that all breast cancer lesions in a single patient express ER that binds TTC-352. The ability to stratify patients using ¹⁸F-TTC352 may streamline advanced TTC-352 clinical trials by removing potential non-responders, thereby increasing the likelihood of demonstrating therapeutic efficacy for the drug.

### Advantages

The disclosure provides a non-invasive imaging and/or identifying of ER+tumors and metastasis in cancer detection and therapy. In particular, the present methods may provide high resolution detection, identification, and localization of breast and lung tumors and metastatic cancer amenable to endocrine and chemotherapy. The compounds disclosed herein (such as ¹⁸F-TTC-352) may function as ligands for ER. The affinity of these ligands for ER is equivalent to estradiol itself at the high affinity end (subnanomolar), or to fluorine-labeled estradiol for other of our ER ligands. Thus, the compounds disclosed herein may lead to new development in personalized cancer treatment, including precision medicine in deadly metastatic cancers.

The present compounds may be used as a new PET tracer that is superior to ¹⁸F-fluoroestradiol (¹⁸F-FES) for visualization of metastatic breast cancer lesions in human and animal models, since the existing agent has significant drawbacks in terms of dosimetry and tumor to background uptake. Peripheral metabolism of ¹⁸F-FES leads to poor localization of metastases in the abdominal region owing to significant drug and drug metabolite uptake in the liver and the intestinal tract (“PET Imaging of Estrogen Receptors as a Diagnostic Tool for Breast Cancer Patients Presenting with a Clinical Dilemma” J. Nucl. Med. 2012, 53, 182)). While bone metastases are imaged effectively using 18F-FES, imaging of ER receptors in soft tissue tumors is extremely difficult. FES is structurally related to a naturally existing hormone and is metabolized in the liver and other organs, causing those organs to exhibit high uptake in PET scans. Thus, while 18F-FES is appropriate for imaging late stage metastatic breast cancer that has already spread to bone, its utility in the staging and localization of early stage breast cancer lesions, and for imaging liver and other abdominal metastases is poor. In contrast, the ¹⁸F-TTC-352 compound disclosed herein is not related to estradiol, and its metabolic breakdown profile may be very markedly different from FES, leading to a much cleaner PET scan image that has much reduced signal in the liver and other organs. This may enable very early detection of metastatic tumors in organs that were previously opaque to PET scans due to the problem with estradiol metabolism. Thus, the radiolabeled tracer compounds disclosed herein may be used in the diagnosis and localization of early stage breast cancer, and as an effective agent to stage early metastatic breast cancer before it has spread to bone.

The disclosed identifying methods may help guide therapy regimens for the treatment of the cancerous lesion. Identification of a cancerous lesion may aid in determining the necessary type of treatment and/or the amount of therapy. For example, estrogen receptors are expressed in several tumor tissues including, but not limited to breast cancer cells. The presence of ER on tumors predicts efficacy of anti-ER cancer drugs including Selective Estrogen Receptor Degraders (SERDs) and aromatase inhibitors. Further, an emerging role for ER expression in chemo-resistant lung cancer indicates that an ER-directed therapeutics may be used in combination with chemo or immune-therapies.

### C. Modes of Administration

As described herein, present compounds or pharmaceutical compositions thereof may be administered to such subjects by a variety of methods. In any of the methods described herein, administration may be by various routes known to those skilled in the art, including without limitation oral, intravenous, intramuscular, topical, subcutaneous, systemic, and/or intraperitoneal administration to a subject in need thereof. In preferred embodiments, the present compounds or compositions are administered by intravenous injection.

The amount of the present compounds or compositions required for imaging and/or diagnostic methods will vary not only with the particular compound or salt selected but also with the route of administration, the nature and/or symptoms of the disease, and the age and condition of the patient, and will be ultimately at the discretion of the attendant physician or clinician. In cases of administration of a pharmaceutically acceptable salt, dosages may be calculated as the free acid or free base.

For parenteral administration such as intravenous injection, the agent can be dissolved or suspended in a physiologically acceptable diluent, such as, e.g., water, buffer, oils with or without solubilizers, surface-active agents, dispersants or emulsifiers. As oils for example and without limitation, olive oil, peanut oil, cottonseed oil, soybean oil, castor oil and sesame oil may be used. More generally spoken, for parenteral administration, the agent can be in the form of an aqueous, lipid, oily or other kind of solution or suspension or even administered in the form of liposomes or nano-suspensions.

The present invention has multiple aspects, illustrated by the following non-limiting examples.

### 6. Examples

Materials and Methods

Reagents. Reagents and solvents were obtained from Sigma-Aldrich (St. Louis, Mo. USA). LC-MS/MS analysis was performed using an API 3000 (Applied Biosystems) triple quadrupole mass spectrometer equipped with Agilent 1200 HPLC (Agilent Technologies, Santa Clara, Calif. USA).

Growth of T47D:A18/PKCα and T47D:A18/neo tumors in vivo. T47D:A18/PKCα and T47D:A18/neo tumors were established as previously described. Tumor cross-sectional area was determined weekly using Vernier calipers and calculated using the formula: length/2×width/2×π. Mean tumor area was plotted against time in weeks to monitor tumor growth. The Animal Care and Use Committee of the University of Illinois at Chicago approved all of the procedures involving animals.

PET imaging using the present radiolabeled compounds or compositions as tracers was conducted in under conditions similar to those previously reported (Kurland et al., Clin. Cancer Res., 2017, 15; 23(2): 407-415). Typically, the compounds or compositions were injected intravenously in a liquid dose of about 1-10 mCi in a volume of about 20 mL isotonic PBS containing less than 15% of ethanol.

Statistical analyses. Statistics were run using GraphPad Prism Version 5.0. Statistical analyses used were one-way ANOVA followed by Tukey's post-test where appropriate.

### Example 1. Synthesis of F18-TTC-352

The starting material for the synthesis was 3-(4-bromophenyl)-6-methoxybenzo[b]thiophene, 1, prepared by previously reported methods such as the processes described in WO2002036584A1, EP778271A2, and W02018081168A2, the contents of which are incorporated herein by reference in their entirety. Compound 1 was converted to 3-(4-bromophenyl)-6-(ethoxymethoxy)-2-(4-(ethoxymethoxy)phenoxy)benzo[b]thiophene, 6, in a manner analogous to that used for preparing fluorinated derivatives in reported studies (US20150284357A1; Xiong R. et al., Selective Human Estrogen Receptor Partial Agonists (ShERPAs) for Tamoxifen-Resistant Breast Cancer, J. Med. Chem. 2016, 59(1):219-37), as shown in Scheme 1 below. Further, compound 6 was converted a diaryliodonium precursor 11 through oxygen-protected intermediates 7-10. While particular oxygen protecting groups (such as ethoxymethoxy group) were used in this example, other suitable oxygen protecting groups known in the art may also be used for the synthesis process described herein to prepare compounds corresponding to 7-11.

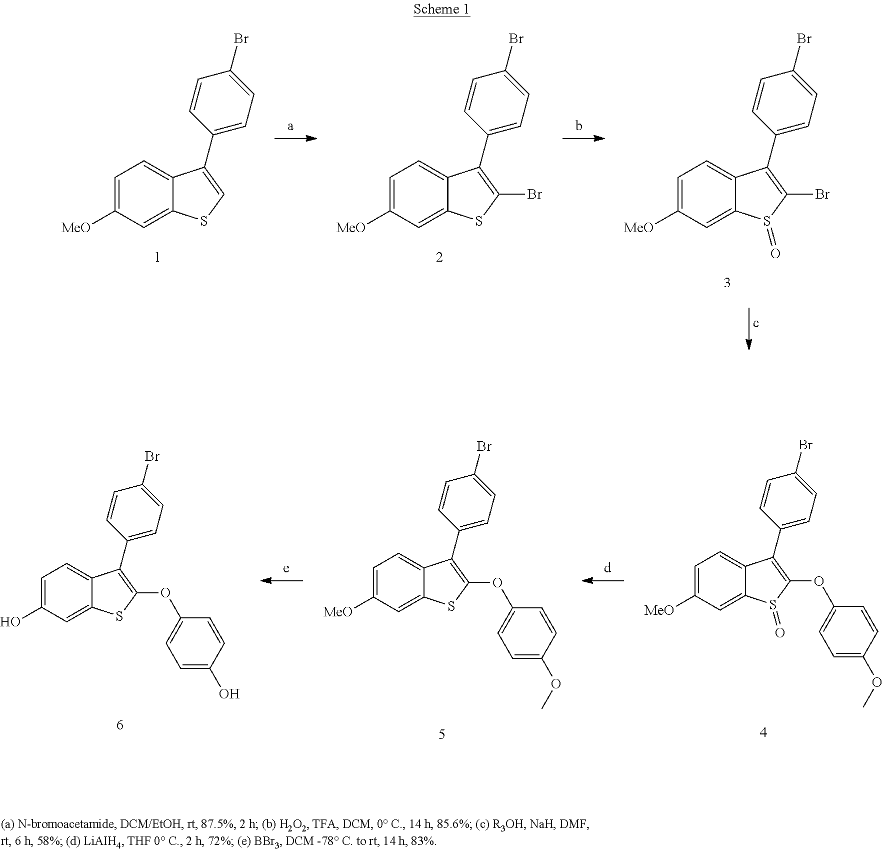 [SMILES: COc1ccc2c(-c3ccc(Br)cc3)csc2c1 ; COc1ccc2c(-c3ccc(Br)cc3)c(Br)sc2c1 ; COc1ccc2c(c1)S(=O)C(Br)=C2c1ccc(Br)cc1 ; Oc1ccc(Oc2sc3cc(O)ccc3c2-c2ccc(Br)cc2)cc1 ; COc1ccc(Oc2sc3cc(OC)ccc3c2-c2ccc(Br)cc2)cc1 ; COc1ccc(OC2=C(c3ccc(Br)cc3)c3ccc(OC)cc3S2=O)cc1]

Preparation of 2-bromo-3-(4-bromophenyl)-6-methoxybenzo[b]thiophene, 2.

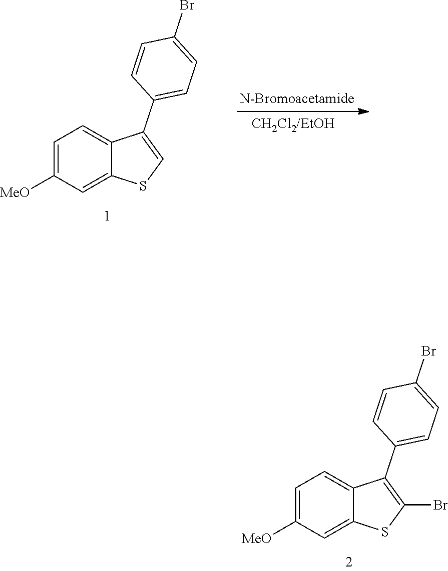 [SMILES: COc1ccc2c(-c3ccc(Br)cc3)csc2c1 ; COc1ccc2c(-c3ccc(Br)cc3)c(Br)sc2c1]

In a 2000 mL flask equipped with a magnetic stir bar, 3-(4-bromophenyl)-6-methoxybenzo[b]thiophene, 1, (29.3755 g, 92 mmol) was dissolved in a solution of DCM (1500 mL) and ethanol (200 mL). N-Bromoacetamide (21.8161 g, 93 mmol) was added in approximately 1 gram portions over the course of an hour, and the mixture was stirred at room temperature for an additional hour. The solvent was removed in vacuo and the remaining solid was washed with absolute ethanol, filtered, and dried to yield 31.90 g (87.5%) of 1 isolated as a fine colorless powder. ¹H NMR (CDCl₃, 400 MHz) δ 7.65-7.63 (m, 2 H), 7.38 (d, J=8.8 Hz, 1 H), 7.36-7.34 (m, 2 H), 7.24 (d, J=2.4 Hz, 1 H), 6.98-9.95 (dd, J=8.2 Hz, 2.4 Hz, 1 H), 3.87 (s, 3 H).

Preparation of 2-bromo-3-(4-bromophenyl)-6-methoxybenzo[b]thiophene 1-oxide, 3.

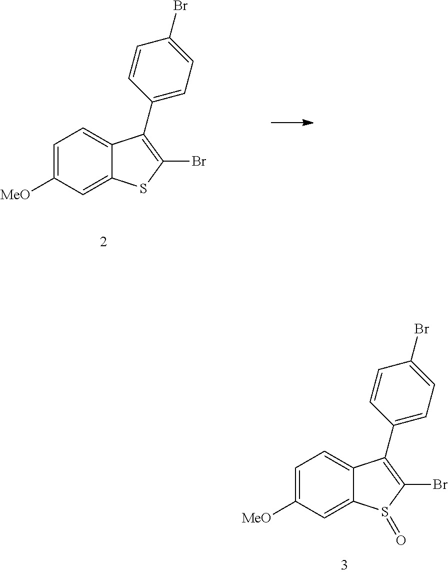 [SMILES: COc1ccc2c(-c3ccc(Br)cc3)c(Br)sc2c1 ; COc1ccc2c(c1)S(=O)C(Br)=C2c1ccc(Br)cc1]

To a stirred, chilled (0° C.) solution of 1 (29.63 g, 74.4 mmol) in 450 mL of anhydrous CH₂Cl₂ trifluoroacetic acid (178 mL) was added dropwise. Aqueous H₂O₂ (7 mL, 80 mmol) was added dropwise, and the resulting mixture was allowed to warm to room temperature and stirred for 14 h. Upon the consumption of starting material, sodium bisulfite (3 g) was added to the solution followed by 50 mL of water. The mixture was stirred for 30 min and then concentrated in vacuo. The residue was partitioned between CH₂Cl₂ and saturated aqueous NaHCO₃ solution. The layers were separated, and the organic layer was washed with water, saturated NaHCO₃, and water and then dried over anhydrous Na₂SO₄. The residue was titrated with diethyl ether and filtered to give 26.66 g (85.6%) of pale yellow powder. ¹H NMR (CDCl₃, 400 MHz) δ 7.69-7.67 (m, 2 H), 7.50 (d, J=2.4 Hz, 1 H), 7.38-7.36 (m, 2 H), 7.15 (d, J=8.2 Hz, 1 H), 6.98-9.95 (dd, J=8.8 Hz, 2.4 Hz, 1 H), 3.89 (s, 3 H)

### Preparation of 3-(4-Bromophenyl)-6-Methoxy-2-(4-Methoxyphenoxy)Benzo[B]Thiophene 1-Oxide, 4.

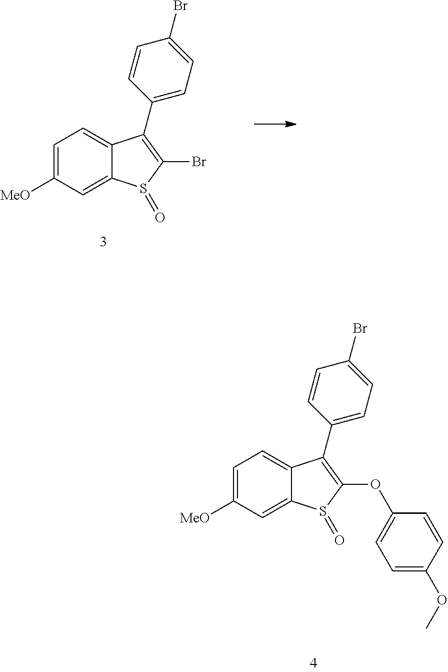 [SMILES: COc1ccc2c(c1)S(=O)C(Br)=C2c1ccc(Br)cc1 ; COc1ccc(OC2=C(c3ccc(Br)cc3)c3ccc(OC)cc3S2=O)cc1]

Under an atmosphere of dry nitrogen, a flame dried 2000 mL Schlenk flask equipped with a magnetic stir bar was charged with 2-bromo-3-(4-bromophenyl)-6-methoxybenzo[b]thiophene 1-oxide, 3 (24.565 g, 59.3 mmol). Anhydrous DMF (1230 mL) was added into the flask by cannula and dissolved compound 3 by continuous stirring. The solution was cooled down to 0° C. by immersing the flask in an ice water bath. A second flamed-dried Schlenk flask equipped with a magnetic stir bar was charged with 4-methoxyphenol (8.839 g, 71.2 mmol) and anhydrous DMF (350 ml). Against positive nitrogen pressure NaH (1.726 g, 71.915 mmol) was added in small portions to the cooled (0° C.) 4-methoxyphenol solution over the course of 2 hours. The thus prepared 4-methoxyphenoxide solution was transferred dropwise by cannula into the solution of 3 over the course of an hour. The resultant reaction mixture was allowed to warm to room temperature and left to stir. The progress of reaction was monitored by TLC, upon conversion of approximately 60% of the starting material to product, the reaction mixture was cooled to 0° C. and quenched by dropwise addition of water. The mixture was transferred to a separatory funnel and extracted with ethyl acetate and water. The organic phase was collected, dried over sodium sulfate, filtered, and evaporated under reduced pressure. The crude organic product was purified by flash chromatography (silica gel, 80:20→70:30 hexanes:ethyl acetate) to obtain 15.722 g of 4 isolated as a yellow powder (yield 58%). ¹H NMR (CDCl₃, 400 MHz) δ 7.58-7.56 (m, 2 H), 7.40 (d, J=2.4 Hz, 1 H), 7.39-7.37 (m, 2 H), 7.24 (d, J=8.8 Hz, 1 H), 7.10-7.06 (m, 2 H), 7.00 (dd, J=8.8 Hz, 2.4 Hz, 1 H), 6.84-6.80 (m, 2 H).

### Preparation 3-(4-Bromophenyl)-6-Methoxy-2-(4-Methoxyphenoxy)Benzo[B]Thiophene, 5.

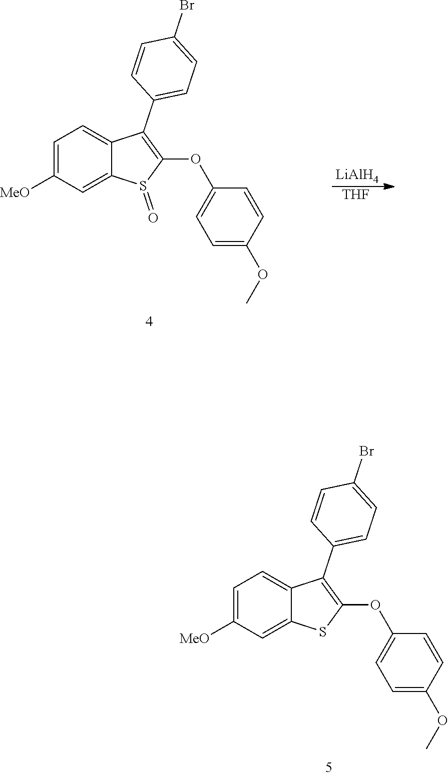 [SMILES: COc1ccc(OC2=C(c3ccc(Br)cc3)c3ccc(OC)cc3S2=O)cc1 ; "LiAlH4" ; COc1ccc(Oc2sc3cc(OC)ccc3c2-c2ccc(Br)cc2)cc1 ; "THF"]

Under an atmosphere of dry nitrogen, a flame dried 1000 mL Schlenk flask equipped with a magnetic stir bar was charged with 3-(4-bromophenyl)-6-methoxy-2-(4-methoxyphenoxy)benzo[b]thiophene 1-oxide, 4 (15.325 g. 33.51 mmol). Anhydrous THF (650 mL) was added to the flask by cannula. After vigorous stirring, a homogenous solution was obtained which was subsequently cooled to 0° C. LiAlH₄ (1.399 g, 36.86 mmol) was weighed out in an inert atmosphere glove box and carefully added to the cooled solution in portions. The solutions was stirred at 0° C. for 2 h and the reaction progress was monitored by TLC. Upon disappearance of the starting material was quenched carefully by the slow addition of 15% aqueous NaOH (1.4 mL) followed by slow addition of water (1.4 mL). The reaction mixture was filtered through celite, and the celite filter pad was washed ethyl acetate (500 mL). The organic fractions were combined, washed with water and brine, dried over sodium sulfate, filtered, and evaporated to dryness. The crude organic product was purified by flash chromatography (silica gel, 95:05→75:25 hexanes: ethyl acetate) to obtain 10.923 g of 5 isolated as a white solid, (yield 72%). ¹H NMR (CDCl₃, 400 MHz) δ¹H NMR (CDCl₃, 400 MHz) δ 7.57-7.55 (m, 2 H), 7.53 (d, J=8.8 Hz, 1 H), 7.44-7.42 (m, 2 H), 7.19 (d, J=2.4 Hz, 1 H), 7.04-7.02 (m, 2 H), 6.97 (dd, J=8.8 Hz, 2.4 Hz, 1 H), 6.84-6.81 (m, 2 H).

Preparation of 3-(4-Bromophenyl)-2-(4-Hydroxyphenoxy)Benzo[B]Thiophen-6-Ol, 6.

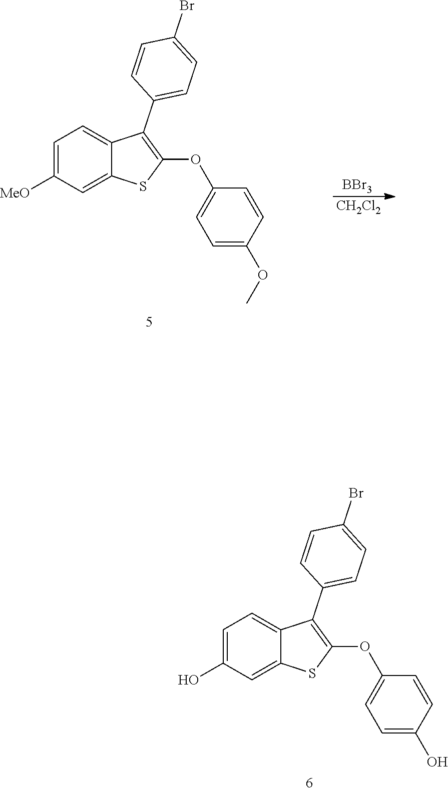 [SMILES: COc1ccc(Oc2sc3cc(OC)ccc3c2-c2ccc(Br)cc2)cc1 ; "CH2Cl2" ; "BBr3" ; Oc1ccc(Oc2sc3cc(O)ccc3c2-c2ccc(Br)cc2)cc1]

Under an atmosphere of dry nitrogen, a flame dried 1000 mL Schlenk flask equipped with a magnetic stir bar was charged with 3-(4-bromophenyl)-6-methoxy-2-(4-methoxyphenoxy)benzo[b]thiophene, 5 (10.923 g. 24.75 mmol). Anhydrous methylene chloride (180 mL) was added to the flask by cannula. After vigorous stirring, a homogenous solution was obtained which was subsequently cooled to −78° C. by immersing the flask in an EtOH and dry ice bath. A sure seal bottle of BBr₃ (Aldrich, 1 M in CH₂Cl₂, used without purification) was pressurized with dry nitrogen and the BBr₃ solution (100 mL, 4 equiv) was transferred to the 1000 mL flask by cannula. The stirred reaction mixture was allowed to warm gradually to room temperature and the reaction progress was monitored by TLC. Upon consumption of all starting material, reaction mixture was cooled to 0° C. and quenched the reaction by dropwise addition of water. The reaction mixture was transferred to a separatory funnel and water (500 mL) was added. After separation of the organic layer, the aqueous layer was extracted twice with ethyl acetate. The organic fractions were combined, washed with water and brine, dried over sodium sulfate, filtered, and evaporated to dryness. The crude organic product was purified by flash chromatography (silica gel, 95:05→75:25 hexanes: ethyl acetate) to obtain 8.31 g white solid, (yield 81.3%). ¹H NMR (CDCl₃, 400 MHz) δ 7.57-7.55 (m, 2 H), 7.50 (d, J=8.8 Hz, 1 H), 7.43-7.41 (m, 2 H), 7.15 (d, J=2.4 Hz, 1 H), 7.00-6.96 (m, 2 H), 6.88 (dd, J=8.8 Hz, 2.4 Hz, 1 H), 6.78-6.74 (m, 2 H).

### Preparation of 3-(4-Bromophenyl)-6-(Ethoxymethoxy)-2-(4-(Ethoxymethoxy)Phenoxy)Benzo[B]Thiophene, 7.

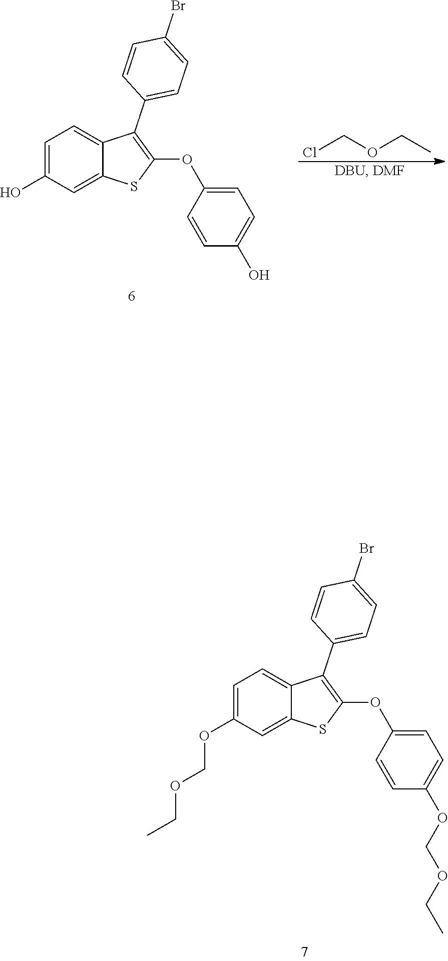

Under an atmosphere of dry nitrogen, a 250 mL round bottom flask equipped with a magnetic stir bar and a rubber septum was charged with 8.31 g (20.123 mmol) of 3-(4-bromophenyl)-2-(4-hydroxyphenoxy)benzo[b]-thiophen-6-ol, 150 mL of anhydrous DMF, and 6.17 mL (50.309 mmol, 2.5 equiv.) of anhydrous 1,8-diazabicyclo[5.4.0]undec-7-ene (DBU). The mixture was stirred at room temperature until a homogenous solution was obtained. Chloromethyl ethyl ether (3.82 ml, 41.253 mmol, 2.05 equiv.) was added dropwise by syringe and the mixture was stirred for 6 hr at room temperature. When TLC analysis (silica gel, 60:40 hexanes : ethyl acetate) indicated that the starting material was consumed and a single product spot was evident, the mixture was poured into aqueous sodium bicarbonate buffer (pH=7) and extracted (3×100 ml) with ethyl acetate. The organic phases were separated, and back extracted (3×100 ml) with buffer solution to remove residual DMF and DBU. The organic phase was separated, dried over sodium sulfate, filtered, and evaporated. The crude reaction mixture was purified by flash chromatography (silica gel, 98:2→90:10 hexanes: ethyl acetate). The progress of the reaction was monitored by TLC. After completion of the reaction, product was extracted by ethyl acetate and purified by flash chromatography (silica gel, 98:2→90:20 hexanes: ethyl acetate). Evaporation of the fractions containing the first visible spot gave the title compound 8.10 g in 76% yield, isolated as a white gummy semisolid material. ¹⁻H NMR (CDCl₃, 400 MHz) δ 7.57-7.55 (m, 2 H), 7.53 (d, J=8.8 Hz, 1 H), 7.43-7.41 (m, 2 H), 7.41 (d, J=2.4 Hz, 1 H), 7.08 (dd, J=8.8 Hz, 2.4 Hz, 1 H), 7.00-6.96 (m, 4 H), 5.26 (s, 2 H), 5.17 (s, 2 H), 3.78-3.70 (q, 4 H), 1.24 (t, 6 H).

### Preparation of 2-(4-(6-(Ethoxymethoxy)-2-(4-(Ethoxymethoxy)Phenoxy)Benzo[B]Thiophen-3-Yl)Phenyl)-4,4,5,5-Tetramethyl-1,3,2-Dioxaborolane, 8.

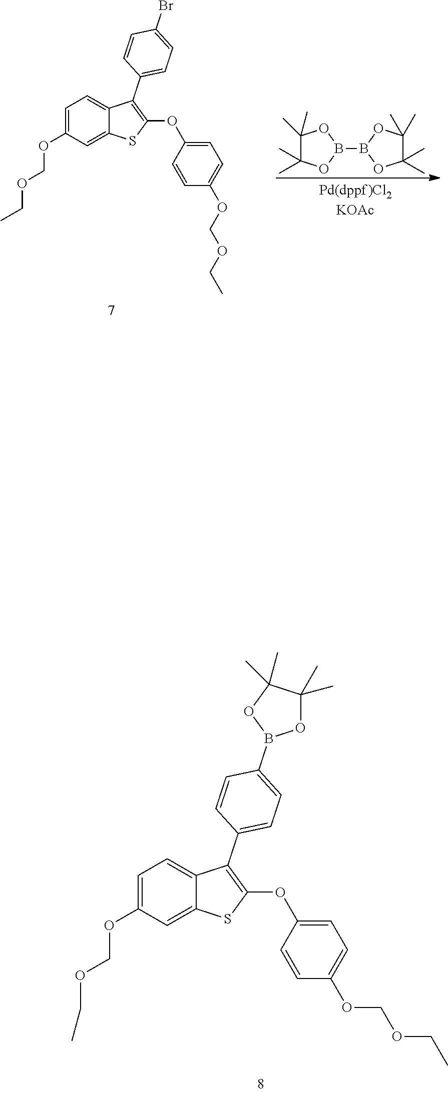

A 250 mL round bottom Schlenk flask equipped with a magnetic stir bar was charged with of 3-(4-bromophenyl)-6-(ethoxymethoxy)-2-(4-(ethoxymethoxy)phenoxy)benzo[b]thiophene, 7, (1 g, 1.89 mmol), 50 mL anhydrous DMSO, potassium acetate (556 mg, 5.7 mmol) and bis(pinacolato)diboron (575 mg, 2.27 mmol). The reaction mixture was stirred for 30 minutes at room temperature under vacuum to degas it. Under an atmosphere of nitrogen, a catalytic amount (˜5 mg) of [1,1′-bis(diphenylphosphino)ferrocene]dichloropalladium(II) (Pd(dppf)Cl₂) was placed in a Schlenk tube, dissolved into 2 mL of anhydrous THF, and treated with 1 drop of n-butyllithium (2.5 M in hexanes). Upon addition of n-butyllithium the solution color changed from orange to cherry red. The catalyst solution was subsequently cannulated into the reaction mixture under nitrogen pressure. The mixture was heated at 80° C. for 3 h with continuous monitoring of progress of reaction by TLC. Upon completion of reaction, the reaction mixture was cooled down at 0° C. and quenched by addition of cold water. The reaction mixture was transferred to a separatory funnel and partitioned between ethyl acetate and water. The organic layer was washed with water and brine, dried over sodium sulfate, filtered and evaporated under reduced pressure. The crude product was purified by flash chromatography (silica gel, 98:2→95:5 hexanes:ethyl acetate) to yield 784 mg of 8 (72% yield) isolated as a white gummy semisolid material. ¹H NMR (CDCl₃, 400 MHz) δ 7.88-7.86 (m, 2 H), 7.59 (d, J=8.8 Hz, 1 H), 7.57-7.55 (m, 2 H), 7.41 (d, J=2.4 Hz, 1 H), 7.06 (dd, J=8.8 Hz, 2.4 Hz, 1 H), 7.00-6.98 (m, 2 H), 7.97-6.95 (m, 2 H), 5.26 (s, 2 H), 5.17 (s, 2 H), 3.78-3.70 (q, 4 H), 1.32 (s, 12 H), 1.24 (t, 6 H).

### Preparation of (4-(6-(Ethoxymethoxy)-2-(4-(Ethoxymethoxy)Phenoxy)Benzo[B]Thiophen-3-Yl)Phenyl)Trifluoro-14-Borane, Potassium Salt, 9.

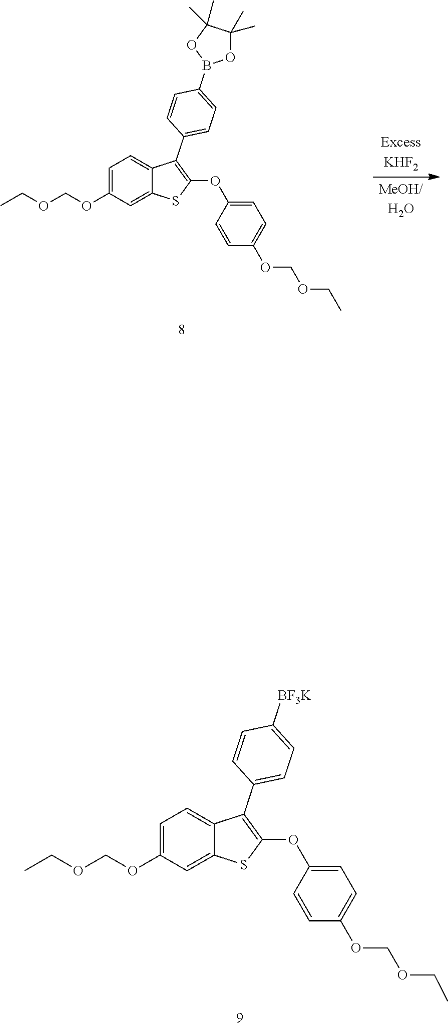

A 250 mL round bottom flask was charged with 8 (800 mg, 1.39 mmol) and dissolved in methanol (80 mL) by continuous stirring and sonication. In a separate beaker, potassium hydrogen fluoride (975 mg, 12.5 mmol) was dissolved in water and added drop wise to the stirred solution in the round bottom flask. The mixture was allowed to stir for 14 h at room temperature before the solvent (methanol and water) was removed under reduced pressure. The product was separated from the inorganic salts by trituration with acetonitrile, followed by filtration to remove potassium bifluoride. Evaporation of acetonitrile gave the colorless product in nearly quantitative yield (98%). ¹H NMR (CDCl₃, 400 MHz) δ 7.59 (d, J=8.8 Hz, 1 H), 7.51-7.49 (m, 2 H), 7.46 (d, J=2.4 Hz, 1 H), 7.32-7.30 (m, 2 H), 7.06 (dd, J=8.8 Hz, 2.4 Hz, 1 H), 7.04-7.01 (m, 2 H), 7.98-6.95 (m, 2 H), 5.26 (s, 2 H), 5.17 (s, 2 H), 3.78-3.70 (q, 4 H), 1.24 (t, 6 H).

### Preparation of (4-(6-(Ethoxymethoxy)-2-(4-(Ethoxymethoxy)Phenoxy)Benzo[B]Thiophen-3-Yl)Phenyl)(4-Methoxyphenyl)Iodonium Trifluoroacetate, 10.

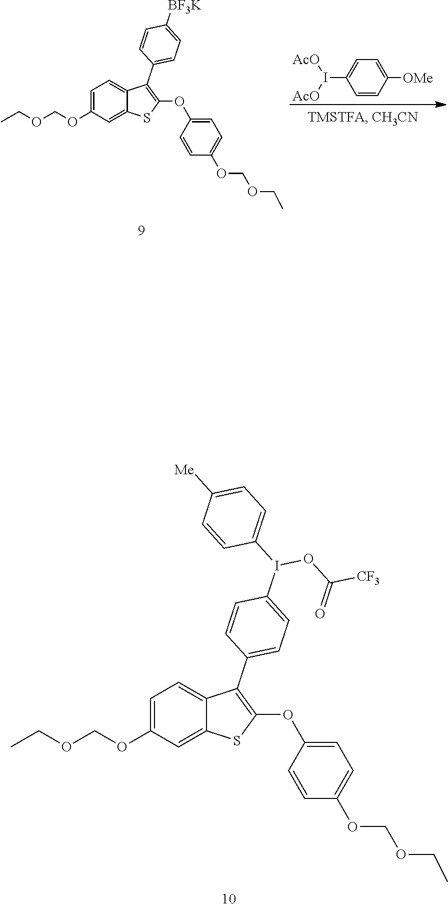

In an inert atmosphere glove box, a 20 mL vial was charged with 9 (381 mg, 0.686 mmol) and 10 mL of anhydrous acetonitrile. In a second vial diacetoxyiodoanisole (253 mg, 0.686 mmol) was dissolved in 5 mL of acetonitrile and the solution was treated with trimethylsilyl trifluoroacetate (129 mg, 0.6932 mmol). The solution was stirred for 10 minutes. The solution containing the activated iodonium salt was added dropwise to the vial containing 9, and the solution was stirred at room temperature for 6 h. The reaction progress was monitored by proton NMR; upon completion of the reaction, solvent was evaporated under reduced pressure and the crude product was washed with pentane (3×5 mL) to remove organic soluble impurities (mainly 4-iodoanisole). The product was dissolved in MTB and precipitated by slow addition of pentane. After 3 consecutive precipitations from MTB and pentane solvent mixture, the off-white solid product (350 mg, 64%) was obtained in >98% purity. ¹H NMR (CDCl₃, 400 MHz) δ 8.10-8.08 (m, 2 H), 8.03-8.01 (m, 2 H), 7.70-7.68 (m, 2 H), 7.50 (d, J=8.8 Hz, 1 H), 7.47 (d, J=2.4 Hz, 1 H), 7.09 (dd, J=8.8 Hz, 2.4 Hz, 1 H), 7.08-6.97 (m, 6 H), 5.25 (s, 2 H), 5.17 (s, 2 H), 3.83 (s, 3 H), 3.73-3.67 (q, 4 H), 1.15 (t, 6 H). ¹H-decoupled-¹⁹F NMR (CDCl₃, 400 MHz) δ −75.36 (s).

### Preparation of (4-(6-(Ethoxymethoxy)-2-(4-(Ethoxymethoxy)Phenoxy)Benzo[B]Thiophen-3-Yl)Phenyl)(4-Methoxyphenyl)Iodonium Trifluoromethylsulfonate, 11.

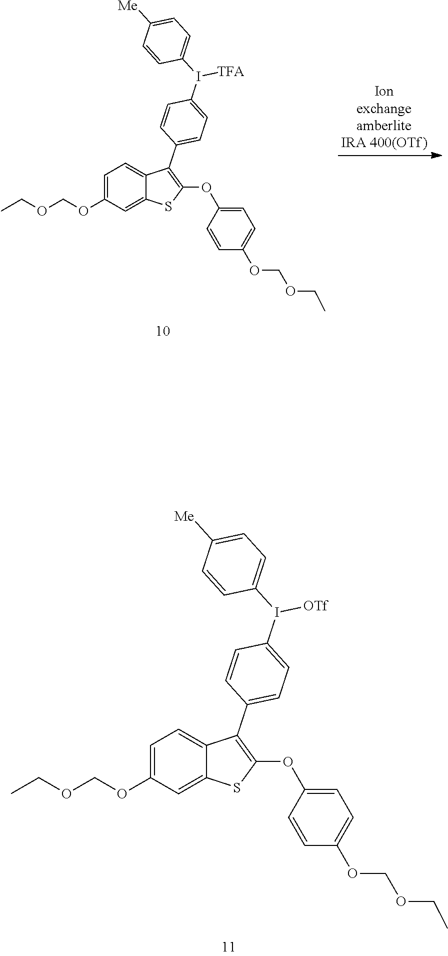

Ion-exchange was performed by dissolving 10 in 10 mL of 20:80 acetonitrile/water. This solution was passed through an Amberlite IRA-400(OTf) ion-exchange column at a rate of 10 drops per minute. Removal of the solvent yielded the triflate salt 11 in quantitative yield. ¹H NMR (CDCl₃, 400 MHz) δ 8.11-8.09 (m, 2 H), 8.04-8.02 (m, 2 H), 7.70-7.68 (m, 2 H), 7.48 (d, J=8.8 Hz, 1 H), 7.45 (d, J=2.4 Hz, 1 H), 7.07 (dd, J=8.8 Hz, 2.4 Hz, 1 H), 7.05-6.95 (m, 6 H), 5.23 (s, 2 H), 5.16 (s, 2 H), 3.83 (s, 3 H), 3.73-3.67 (q, 4 H), 1.15 (t, 6 H). ¹H-decoupled-¹⁹F NMR (CDCl₃, 400 MHz) δ −79.19 (s, decoupled with hydrogen).

### Preparation of ¹⁸F-3-(4-(Fluoro)Phenyl)-2-(4-Hydroxyphenoxy)Benzo[B]Thiophen-6-Ol, 12 (18F-TTC-352).

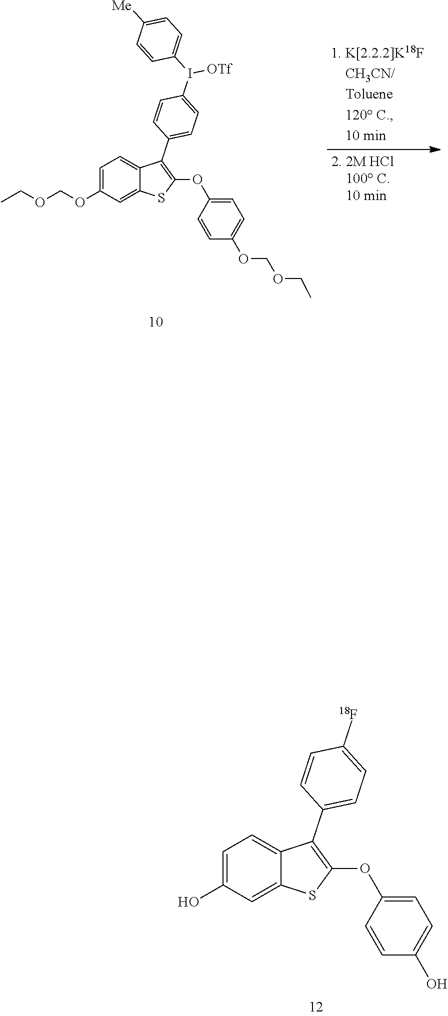 [SMILES: CCOCOc1ccc(Oc2sc3cc(OCOCC)ccc3c2-c2ccc(I(OS(=O)(=O)C(F)(F)F)c3ccc(C)cc3)cc2)cc1 ; Oc1ccc(Oc2sc3cc(O)ccc3c2-c2ccc([18F])cc2)cc1]

The purified precursor 11 and organic solvents for radiosyntheses were stored in an anaerobic chamber under argon to reduce exposure to water in the atmosphere. ¹⁸F-3-(4-(fluoro)phenyl)-2-(4-hydroxyphenoxy)benzo[b]thiophen-6-ol, 12, was produced in a two-step synthesis by thermolysis and subsequent deprotection of a diaryliodonium fluoride adapted for preparation on a Synthera® automated synthesizer (IBA) using an Integrated Fluidic Processor™ (ABX) and an automated script.

Aqueous [¹⁸F]fluoride produced in an IBA Cyclone® 18/9 cyclotron from 97% ¹⁸O-enriched water was passed through a QMA cartridge (ORTG, Inc., pretreated with 2 mL of 1 M NaHCO₃ and rinsed with 5 mL water) to trap the [¹⁸F]fluoride while the [¹⁸O]water passed through to waste. The [¹⁸F]fluoride was eluted from the QMA resin with 620 μL of a 94:6 CH₃CN:H₂O solution containing K₂CO₃ (0.62 mg) and Kryptofix [2.2.2.] (6.2 mg). The solvent was removed by azeotropic distillation under argon flow with reduced pressure and heating to 110° C. for 2.5 minutes. The diaryliodonium salt precursor 11 (6 mg) dissolved in 10% anhydrous CH₃CN and toluene (1 mL) was added to the reactor and the mixture was heated to 120° C. for 8 minutes. The solvent was removed under reduced pressure with argon flow (approx. 3 minutes) and deprotection of the fluorinated complex was achieved by addition of 800 μL of 2 M HCl and heating to 100° C. for 10 minutes to afford the crude [¹⁸F]-labeled product. The acidic solution was diluted by the addition of 4 mL of HPLC eluent and then passed through a 0.45 μm filter to a 5 mL HPLC loop. Purification was achieved by semi-preparative HPLC utilizing a Hamilton PRP-1 column (10×250 mm, 10 μ) with an eluent of 10% ethanol and 28 mM HCl, 20 mM ammonium acetate, 0.04% ascorbic acid (pH 2) at a flow rate of 3.5 mL/min. By monitoring with a radioactivity detector, the desired product was collected in a 7-9 mL fraction. Neutralization of the final product was achieved by the addition of 2 mL of 0.113 M ammonium acetate (pH 8.2). Sterile water was also added (1:4, v/v) to dilute the total ethanol concentration to below 2%. This method produced 12 (RCY=10.5±3.5% EOS, n=9) in >99% radiochemical purity and a total synthesis time of 56±2 min.

Assessing the impact of precursor on labeling and specific activity.

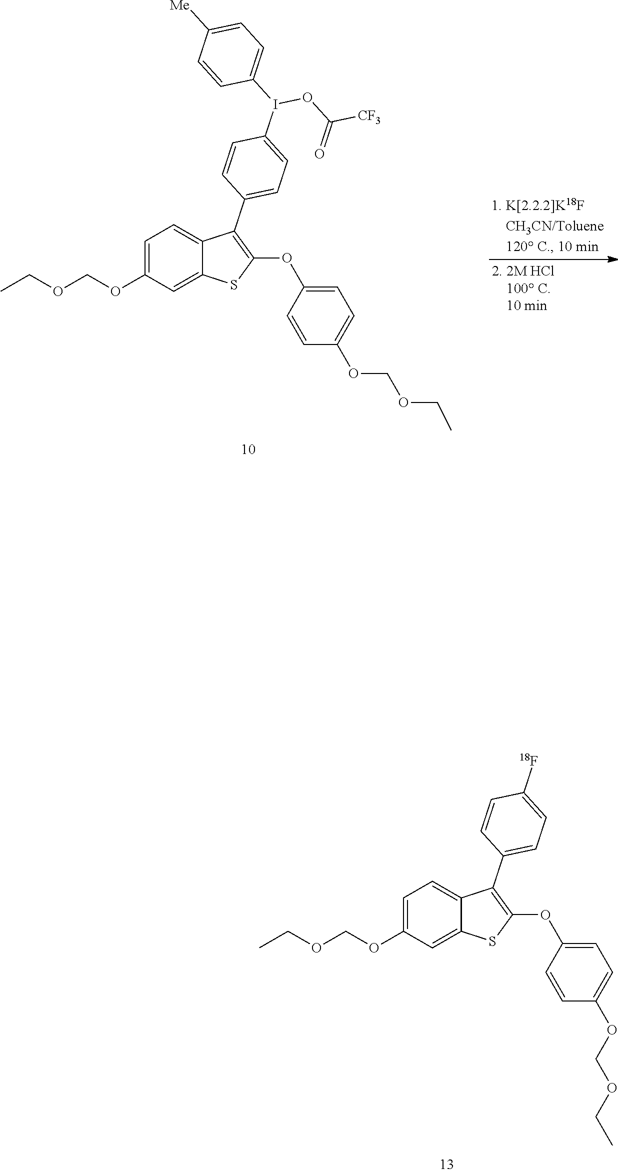

To test the impact of fluorinated counterion upon the specific activity of the isolated product, radiosynthesis of 13 was also performed using the trifluoroacetate salt precursor, 10. The diaryliodonium salt precursor 10 (6 mg) dissolved in 10% anhydrous CH₃CN and toluene (1 mL) was added to the reactor and the mixture was heated to 120° C. for 8 minutes in the presence of dry Kryptofix [2.2.2]K¹⁸F, prepared as described above. The solvent was removed under reduced pressure with argon flow (approx. 3 minutes). This material was assessed by analytical radioHPLC. Radiochemical purity was confirmed by analytical HPLC using an Agilent Zorbax SB-Aq column (4.6×150 mm, 5 μm) with an eluent of 10% acetonitrile and 35 mM phosphoric acid, 25 mM monobasic sodium phosphate, pH 2 at a flow rate of 1 mL/min with monitoring at 220 nm. If present, the area of the UV peak with a retention time corresponding to the standard solution of [¹⁹F]13 of known concentration was compared to the area of that standard. From this, the total mass of 13 was calculated. No discernable difference in yield or specific activity was found when labeling was performed with either precursor. Labeling yields were 15-20% and specific activity was ≤0.5 Ci/uM.

### Example 2 Animal Imaging and PET Probe Validation

Animal imaging study may be carried out: 1) to confirm that a PET probe (such as ¹⁸F-TTC-352) may be selectively taken up by ER+ breast cancer tumors, 2) to determine the extent of tumor to background uptake, and 3) to confirm that tumor response to treatment may be successfully imaged by the novel radiotracers.

PET probes may be synthesized by the representative synthesis process described in Example 1 above. The PET probes may be transferred for imaging using a PET scanner. Radiotracer injection and imaging may be performed in a PET facility.

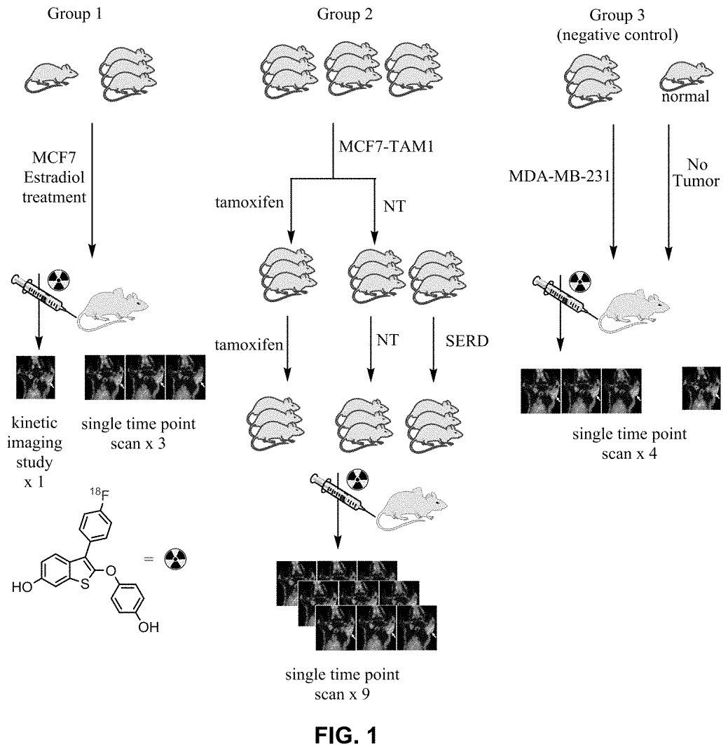

Breast cancer xenograft animal imaging study. PET imaging may be performed to validate ER signal intensity and specificity in three groups of tumor-bearing mice. All animal handling may be carried under an approved IACUC protocol. As shown in FIG. 1, Group 1 imaging studies may examine ¹⁸F-TTC-352 uptake in 4 athymic ovariectomized mice implanted with MCF7 cells (bilateral tumor injection, 20 million MCF7 cells) treated with estradiol. Tumor volume may be measured weekly with Vernier calipers, and after tumor volume reaches 400 mm3, the mice may be injected intravenously in the tail vein with an isotonic solution of the radiotracer (50 μL, ˜200 uCi) in saline. One mouse may be imaged repeatedly (30 min, 1 h, 1.5 h) to determine an optimal imaging time point, and all remaining mice may be imaged at the selected post-injection time point. Group 2 mice may be implanted with MCF7-TAM1 cells into the mammary fat pads of 9 athymic nude ovariectomized female mice (4-7 weeks old). These tumors are tamoxifen resistant and grow in the absence of estradiol supplementation. Tumors may be allowed to grow in the presence of tamoxifen (daily oral gavage) (3 mice); or not treated (6 mice NT) as previously described. Tumors from both treatment groups are expected to reach a volume of approximately 400 mm3 in 4-6 weeks. At that time three mice from the NT group may be administered a Selective Estrogen Receptor Downregulator (SERD) by oral gavage. SERDs cause complete degradation of the ER and based on previous results21, tumors may regress by approximately 50% within 8-12 days. At this point, all 9 mice (3 tamoxifen; 3 NT; 3 SERD treated) may be transferred for PET imaging. In Group 3, the ER negative xenograft model, MDA-MB-231 and a mouse without tumor may serve as negative controls to investigate ER specificity and background uptake.

A PET scan may be carried out on a C57BL/6 mouse administered with ¹⁸F labeled probes disclosed herein. The probe may be dissolved in a solvent and administered to the mice through tail vein injection of 100 to 300 microcuries of the compound in a suitable solvent. Suitable solvents may include dimethyl sulfoxide (DMSO, 5%), 4% 1:1 Solutol:dimethyl acetamide (DMA), and ethanol (8%). The specific activity of the probe may be about 0.1 to about 1.0 Ci/μmol. A 2-hour dynamic PET scan of the mice may be conducted, and the distribution of the probe in various organs of the mice may be monitored. In particular, the radioactivity in organs such as bladder, intestines, liver, and lung may be determined in about 1 to about 15 minutes after injection. The results from mice having cancer may be compared to healthy controls.

### Example 3 Representative Development of an ER Ligand for PET Scan

Phase I. Synthetic procedures may be carried out to obtain 95% purity with high speed and efficiency in the cold (19F) fluorine incorporation in at least two ER ligands with affinity/potency at ERα equivalent to fluoroestradiol. Pharmacokinetic analysis may be conducted using cold probes in rats for plasma, brain, and liver distribution, including glucuronides. Further studies may be conducted in NSG mice, imaging MCF-7 metastasis as in Ogba, N.; Manning, N. G.; Bliesner, B. S.; Ambler, S. K.; Haughian, J. M.; Pinto, M. P.; Jedlicka, P.; Joensuu, K.; Heikkila, P.; Horwitz, K. B. Luminal breast cancer metastases and tumor arousal from dormancy may be promoted by direct actions of estradiol and progesterone on the malignant cells (Breast Cancer Res 2014, 16, 489). The lead compounds may be compared to ¹⁸F-FES.

Phase II. 1. Automated radiosynthesis for the best two ¹⁸F-labeled ER PET probes identified in Phase I may be carried out. Commercial benchmarks for the automated synthesis are a) <90 min synthesis time, b) specific activity of the final product greater than 1 Ci/umol, c) radiochemical purity>98%, d) chemical purity>95%, and e) radiochemical yield>25%. A Chemistry, Manufacturing and Controls (CMC) data section may be completed to support an Investigational New Drug (IND) application to FDA. Fully controlled model imaging study may be conducted in ER+ and ER− breast tumor bearing mice, including competitive displacement study with estradiol.

As an example, TTC-352 and other benzothiophene SERMs (e.g., those described in WO2014066692A1 and WO2014066695A1) are potent estrogen receptor ligands in vitro and possess “drug-like” physicochemical properties that make them attractive as potential imaging agents. The performance of such ligands in imaging applications may depend on their biodistribution, metabolism, and clearance characteristics. Preferably, a fluorinated PET probe may localize only in the tissues of interest, undergo little metabolism, and clear rapidly to provide a low-background image with little radiation exposure to the patient. Off-target uptake and extensive metabolism are, in fact, the problems associated with the current PET probe for estrogen receptor imaging, ¹⁸F-FES. The radiosynthesis of promising ER-PET ligands may be carried out as described above, and the pharmacokinetics of these ligands may be measured to demonstrate their significant advantages over ¹⁸F-FES.

After completion of the Phase II studies, human imaging may be carried out under an IND protocol, including using PET probes into clinical studies. Clinical translation of fluorinated PET tracers may also be conducted.

Various changes and modifications to the disclosed embodiments will be apparent to those skilled in the art. Such changes and modifications, including without limitation those relating to the chemical structures, substituents, derivatives, intermediates, syntheses, compositions, formulations, or methods of use of the invention, may be made without departing from the spirit and scope thereof

What is claimed is:

1. A radiolabeled compound of formula (I) 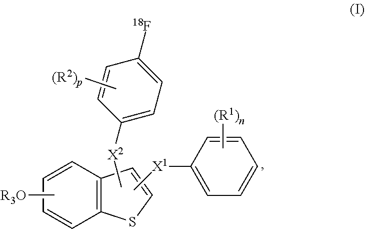
or a pharmaceutically acceptable salt thereof, wherein
X¹ is a bond, —O—, or —C(O)—;
X² is a bond, —O—, or —C(O)—;
R¹ at each occurrence is independently C₁₋₄ alkyl, C₁₋₄haloalkyl, halogen, —CN, —C(O)OR^{1a}, —(CH═CH)—C(O)OR^{1a}, —OR^{1a}, —NR^{1b},R^{1c}, or —SO₂CH₃,
R² at each occurrence is independently C₁₋₄ alkyl, halogen, —OR^{2a}, or deuterium;
R³ is hydrogen or C₁₋₄ alkyl;
R^{1a}, R^{2a} at each occurrence are independently hydrogen, C₁₋₄ alkyl, or C₁₋₄alkylene-G¹;
R^{1b}, R^{1c} at each occurrence are independently hydrogen or C₁₋₄ alkyl;
G¹ is phenyl, a 5- or 6-membered heteroaryl, or a 5- or 6-membered heterocycle, wherein the phenyl, heteroaryl, and heterocyle are each optionally substituted with 1, 2, 3, 4, or 5 substituents independently selected from the group consisting of C₁₋₄alkyl, C₁₋₄haloalkyl,—OC₁₋₄alkyl, —C(O)—C₁₋₄alkyl, and halogen;
n is 0, 1, 2, 3, 4, or 5; and
p is 0, 1, 2, 3, or 4.

2. The compound of claim 1, or a pharmaceutically acceptable salt thereof, having formula (I-a)

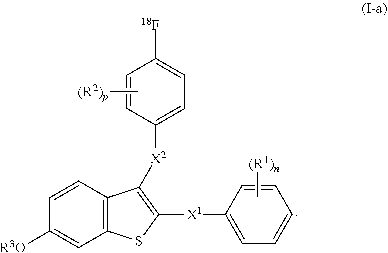

3. The compound of claim 2, or a pharmaceutically acceptable salt thereof, having formula (I-a-1) 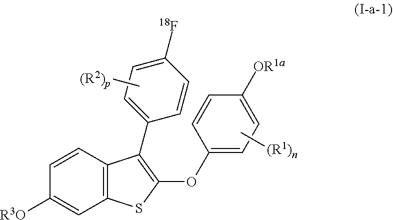
wherein n is 0, 1, 2, 3, or 4.

4. The compound of claim 3, or a pharmaceutically acceptable salt thereof, having formula (X) 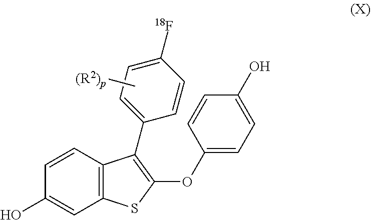
wherein R² is deuterium.

5. The compound of claim 4, or a pharmaceutically acceptable salt thereof, which is

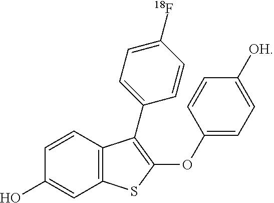 [SMILES: Oc1ccc(Oc2sc3cc(O)ccc3c2-c2ccc([18F])cc2)cc1]

6. The compound of claim 1, or a pharmaceutically acceptable salt thereof, having a radiochemical purity of at least 90%.

7. The compound of claim 1, or a pharmaceutically acceptable salt thereof, having a radiochemical purity of at least 99%.

8. A pharmaceutical composition comprising a compound of claim 1, or a pharmaceutically acceptable salt thereof, and a pharmaceutically acceptable carrier.

9. A method of imaging a tissue expressing estrogen receptor, comprising:
administrating to a subject having an estrogen-related medical disorder a diagnostically effective amount of a radiolabeled compound of claim 1, or a pharmaceutically acceptable salt thereof; and
detecting the radioactivity of the radiolabeled compound, or the pharmaceutically acceptable salt thereof, in the tissue of the subject.

10. The method of claim 9, wherein the estrogen-related medical disorder is selected from the group consisting of: cancer, inflammation, osteoporosis, vaginal atrophy, central nervous system diseases, and cardiovascular system diseases.

11. The method of claim 10, wherein the cancer is selected from the group consisting of breast cancer, ovarian cancer, prostate cancer, and lung cancer.

12. The method of claim 11, wherein the cancer is a breast cancer.

13. The method of claim 12, wherein the breast cancer is ER-positive breast cancer.

14. The method of claim 12, wherein the breast cancer is resistant to ER-targeted endocrine therapy.

15. The method of claim 14, wherein the breast cancer is tamoxifen resistant breast cancer.

16. The method of claim 9, wherein the radioactivity is detected by positron emission tomography (PET).

17. The method of claim 9, wherein the radiolabeled compound is 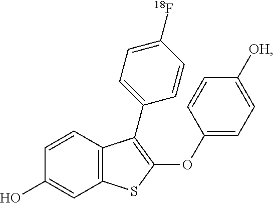 [SMILES: Oc1ccc(Oc2sc3cc(O)ccc3c2-c2ccc([18F])cc2)cc1]
or a pharmaceutically acceptable salt thereof.

18. A method of identifying cancer lesion, comprising:
(a) administrating to a subject having cancer a diagnostically effective amount of a radiolabeled compound of claim 1, or a pharmaceutically acceptable salt thereof and
(b) detecting the radioactivity of the radiolabeled compound, or the pharmaceutically acceptable salt thereof, in an organ of the subject;
wherein if the radioactivity in (b) is greater than a radioactivity in a same organ from a subject not having cancer, then the radioactivity in (b) indicates cancer lesion in the organ.

19. The method of claim 18, wherein the cancer lesion is a result of cancer metastasis.

20. The method of claim 19, wherein the cancer metastasis is localized in bone, brain, chest wall, liver lymph, or lung.

21. The method of claim 18, wherein the cancer is breast cancer.

22. The method of claim 21, wherein the breast cancer is ER-positive breast cancer.

23. The method of claim 18, wherein the cancer is a recurrent cancer.

24. The method of claim 18, wherein the radioactivity is detected by positron emission tomography (PET).

25. The method of claim 18, wherein the radiolabeled compound is 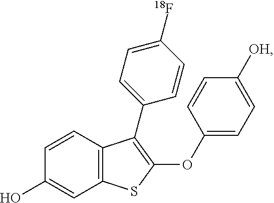 [SMILES: Oc1ccc(Oc2sc3cc(O)ccc3c2-c2ccc([18F])cc2)cc1]
or a pharmaceutically acceptable salt thereof
